(19) United States
(12) Patent Application Publication
(10) Pub. No.: US 2018/0280545 A1
(43) Pub. Date: Oct. 4, 2018

(54) IRON OXIDE NANOPARTICLES AND THEIR SYNTHESIS BY CONTROLLED OXIDATION

(71) Applicant: University of Washington, Seattle, WA (US)

(72) Inventors: Kannan M. Krishnan, Seattle, WA (US); Amit Praful Khandhar, Seattle, WA (US); Richard M. Ferguson, Seattle, WA (US); Scott Jeffrey Kemp, Seattle, WA (US)

(73) Assignee: University of Washington, Seattle, WA (US)

(21) Appl. No.: 15/524,589

(22) PCT Filed: Oct. 30, 2015
(86) PCT No.: PCT/US2015/058425

§ 371 Date: May 4, 2017

Related U.S. Application Data
(60) Provisional application No. 62/074,820, filed on Nov. 4, 2014.

(51) Int. Cl. A61K 49/18; A61B 5/05; A61B 5/055; A61F 7/00; G01R 33/56; G01R 33/12

(52) U.S. Cl. CPC A61K 49/183; A61B 5/0515; A61B 5/055; A61F 2007/009; G01R 33/5601; G01R 33/1276; A61F 7/00

(57) ABSTRACT

Disclosed herein are iron oxide nanoparticles having an iron (II) content in a metastable state that is intermediate the iron (II) content of wüstite and magnetite. The disclosed iron oxide nanoparticles exhibit unexpectedly beneficial magnetic properties (e.g., saturation magnetization) resulting from both the size of the nanoparticles and the iron (II) content. Accordingly, the iron oxide nanoparticles are attractive for magnetic imaging applications, such as magnetic particle imaging. Methods of forming the iron oxide nanoparticles are also provided, such methods including a controlled oxidation step wherein a small amount (e.g., 1%) of gaseous oxygen is exposed to wüstite nanoparticles for a defined period of time sufficient to partially oxidize the wüstite but prevent conversion entirely to magnetite. Finally, methods of using the iron oxide nanoparticles are also provided. Representative methods include magnetic particle imaging, magnetic resonance imaging, and hyperthermia.

### CROSS-REFERENCE TO RELATED APPLICATION

This application claims the benefit of U.S. Patent Application No. 62/074,820, filed Nov. 4, 2014, the disclosure of which is hereby incorporated by reference in its entirety.

### STATEMENT OF GOVERNMENT LICENSE RIGHTS

This invention was made with Government support under 1R42EB013520-02 awarded by National Institutes of Health. The Government has certain rights in the invention.

### BACKGROUND

Iron oxide nanoparticles are desirable magnetic imaging agents due to their exceptional magnetic properties. For example, ferumoxytol (Feraheme) is a magnetic resonance imaging (MRI) contrast agent and comprises magnetite nanoparticles.

Iron oxides are complex group of materials that have structure-dependent properties that are still not yet fully understood. Depending on how the nanoparticles are formed, their character may be one or more of the many different iron oxide species, including:
Iron(II) oxide, wüstite (FeO)
Iron(II,III) oxide, magnetite (Fe₃O₄)
Iron(III) oxides (Fe₂O₃)
   alpha phase, hematite (α-Fe₂O₃)
   beta phase, (β-Fe₂O₃)
   gamma phase, maghemite (γ-Fe₂O₃)
   epsilon phase, (ε-Fe₂O₃).

In one known method, monodisperse wüstite iron oxide nanoparticles are formed through thermolysis at temperatures of 250° C. to 360° C. of an iron-containing compound, such as iron oleate or iron pentacarbonyl, using a high boiling organic solvent such as octadecene and often adding a “surfactant” such as oleic acid, to form iron(II) oxide wüstite (FeO) nanoparticles. Subsequent to thermolysis an oxidation step can be used to form nanoparticles of different iron oxide species, such as iron (II,III) oxide, magnetite (Fe₃O₄) and thus tailors their physical properties as a result of the iron oxide species contained within the nanoparticle.

Despite increasing commercial uses for iron oxide nanoparticles, the present oxidizers known to those of skill in the art have certain hazards associated with their use for the oxidation of iron based nanoparticles, as follows.

To oxidize iron based nanoparticles four reagents have been employed. These include sodium hypochlorite, m-Chloroperbenzoic acid (mCPBA), trimethylamine N-oxide and air (˜20% oxygen).

The use of air (or ˜20% oxygen) for the oxidation of iron oxide nanoparticles has been reported numerous times, all on very small scale. On larger scale the dangers of catastrophic fire is a major concern to the safe operation of manufacturing facilities. Therefore, the use of air or 20% oxygen in combination with flammable solvents at elevated temperatures such as 1-octadecene (flash point, 155° C. and autoignition temperature, 250° C.) is very hazardous due to the risk of fire. To use air safely the flammable reaction mixture would need to be lowered from the particle formation temperature, typically >300° C., to a temperature much less than the autoignition temperature of the solvent and preferably less than the flash point of the solvent.

The use of mCPBA is incompatible with solvents and reagents containing olefins, such as 1-octadecene and oleic acid, due to the epoxidation of olefins with mCPBA (“MCPBA Epoxidation of Alkenes: Reinvestigation of Correlation between Rate and Ionization Potential” Cheal Kim, Teddy G. Traylor, and Charles L. Perrin J. Am. Chem. Soc. 1998, 120, 9513-9516). Additionally, the use of a strong oxidizier, like mCPBA, is hazardous because the risk of fire and explosions.

The use of trimethylamine N-oxide poses health risk and trimethylamine N-oxide is classified as a hazardous substance by OSHA (OSHA 29 CFR 1910.1200). Additionally trimethylamine N-oxide is a strong oxidizer which is considered a fire hazard. In the process of oxidation using trimethylamine N-oxide, the by-product trimethylamine is produced. Trimethyl amine has a flash point of −7° C. and a boiling point of 4° C. which would require special handling and recovery procedures and equipment at larger scale.

The use of aqueous sodium hypochlorite to oxidize nanoparticles is limited for the oxidation of hydrophobic nanoparticles such as the oleic acid coated iron oxide nanoparticles produced by the thermal decomposition of iron oleate, because of the insolubility of these iron oxide nanoparticles in aqueous media.

There is a need for oxidation conditions, which minimize use of toxic materials, avoid use of possibly explosive reagents and reduce the risk of fire.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, a plurality of iron oxide nanoparticles is provided. In one embodiment, each iron oxide nanoparticle includes a core comprising an iron oxide inner core comprising an intermediate phase between stoichiometric FeO and stoichiometric Fe₃O₄, the inner core comprised of iron (II) in an amount of from 34% to 50% of the total iron in the inner core.

In a second aspect, a plurality of iron oxide nanoparticles is provided, each iron oxide nanoparticle comprising a core of iron oxide, wherein the plurality of iron oxide nanoparticles has an iron (II) content of 25-37% of the total iron content; and wüstite (FeO) is visible in a select area electron diffraction image obtained by transmission electron microscopy.

In another aspect, a plurality of iron oxide nanoparticles is provided, wherein each iron oxide nanoparticle includes a core comprising an iron oxide inner core comprising an intermediate phase between stoichiometric FeO and stoichiometric Fe₃O₄, the inner core comprised of iron (II) in an amount of from 34% to 50% of the total iron in the inner core; wherein each iron oxide nanoparticle has a total iron (II) content of 25-37% of the total iron content; and wüstite (FeO) is visible in a select area electron diffraction image obtained by transmission electron microscopy.

In another aspect a method is provided that comprises applying a magnetic field to a plurality of iron oxide nanoparticles as disclosed herein.

In one aspect, a method of forming a plurality of iron oxide nanoparticles is provided. In one embodiment, the method includes:

forming a plurality of iron oxide nanoparticles in a solution using thermolysis; and

oxidizing the plurality of iron oxide nanoparticles in the solution by exposure to a gas mixture comprising an inert gas and a gaseous oxygen (O₂) content of from 0.01 to 5% by volume to provide a plurality of oxidized iron oxide nanoparticles;

wherein the step of oxidizing the plurality of iron oxide nanoparticles takes place at an oxidation temperature of 200° C. to 370° C.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1A: Wüstite nanoparticles obtained by thermolysis under argon for 2.5 hours at 324° C. without an oxidation step.

FIG. 1B: Selected area electron diffraction (SAED) pattern (and radial integration, shown in FIG. 1C)) Indexed rings correspond to wüstite unless noted. The magnetite rings are notably more diffuse, in contrast to the sharp wüstite rings, suggesting small magnetite crystallites due to oxidation of a thin shell on the particle surface.

FIGS. 2A-2J: Nanocrystal evolution during the thermolysis reaction. Bright field TEM images of sample aliquots (FIGS. 2A-2D), with selected area electron diffraction (FIGS. 2E-2H). Note that FIGS. 2D and 2H represent nanoparticles after the oxidation step.

FIG. 21 graphically illustrates time evolution of particle size measured by TEM and DLS. DLS data is not shown for the final oxidized sample, since the strongly magnetic particles aggregated. FIG. 2J graphically illustrates shows magnetization curves measured at 295 K.

FIGS. 3A-3F. Bright field TEM images of nanoparticles with varying diameter. FIG. 3A: 15 nm, FIG. 3B: 20 nm, FIG. 3C: 24 nm, FIG. 3D: 27 nm, FIG. 3E 30 nm, and FIG. 3F 35 nm.

FIGS. 4A-4C. Selected area electron diffraction of nanoparticles oxidized in octadecene (FIG. 4A) and octadecane (FIG. 4B), and (FIG. 4C) radial integrations of the diffraction patterns (sampling 40° centered on 270°). FIG. 4B features unique diffraction rings 2.7 and 2.9 l/nm, which match reference patterns for maghemite.

FIG. 5. Graphically illustrates the relationship between saturation magnetization and core Fe(II)%. These properties are correlated, with the saturation magnetization growing as the Fe(II)% decreases while the nanoparticle oxidize from Wüstite to Magnetite.

FIGS. 6A-6C: Evolution of differential susceptibility, χ(H) with oxidation for nanoparticles of different size, measured at 25 kHz applied field, 20 mT/μ0 amplitude.

FIG. 6A shows results for particles with cores between 33 and 37% Fe(II); FIG. 6B shows results for cores with 37-38% Fe(II); and FIG. 6C shows inner cores with 39-40% Fe(II). Top portions of FIGS. 6A-6C are normalized for comparing the peak width, (Δ), while bottom portions of FIGS. 6A-6C show intensity per unit iron.

FIGS. 7A-7D: Summary results of intensity of χ, the maximum value of χ(H), here normalized by χRes, the value measured for Resovist, a commercial nanoparticle, for a range of sample compositions, presented with Fe(II)% (FIGS. 7A and 7B) and Ms (FIGS. 7C and 7D) used to characterize the composition. FIG. 7B shows a slice of the full set with narrow range of diameters, indicating the trend apparent.

FIG. 8A-8D: Results for Δ (FWHM of χ(H), (at 25 kHz, 20 mT/μ₀). Each bar represents a unique sample. Here, smaller Δ represents better performance. Measured values were normalized by ΔRes, the value measured for Resovist. FIGS. 8A and 8B show performance versus size, and Fe(II)%, with B being a narrow slice of diameter for emphasis. FIGS. 8C and 8D plot performance vs size and saturation magnetization, Ms, with D showing a narrower range of Mₛ for emphasis. In FIG. 8A, note the cluster of samples around 25-28 nm and ˜40% Fe(II), which displayed the most desirable behavior. In FIGS. 8C and 8D, the favored region is Ms<400, and size between 23 and 28 nm.

FIGS. 9A and 9B. We define a Figure of Merit, χ/Δ, to be the peak intensity of χ(H) divided by FWHM. The figure of merit captures both relevant parameters and presents them so that the greatest value (tallest bar) represents the most desirable behavior. In FIG. 9A, the cluster of tall bars is centered around 40% Fe(II), with sizes between 24 and 28 nm. For example, nanoparticles larger than 28 nm in diameter have performance decreased with increasing size when Fe(II) was 40%. In FIG. 9B, characteristics of the same nanoparticle samples are presented, except using saturation magnetization, Mₛ, to describe the iron composition instead of titrated iron (II) %. The best example nanoparticles had Mₛ less than 400 kA/m but greater than 350.

FIGS. 10A-10J are bright-field TEM images and selected area electron diffraction images for examples of iron oxide nanoparticles that were titrated to determine their iron (II) % and FIG. 10K illustrates radial integrations for each of the diffraction patterns.

### DETAILED DESCRIPTION

Disclosed herein are iron oxide nanoparticles tailored to have an iron (II) content in a metastable state that is intermediate the iron (II) content of wüstite and magnetite. The disclosed iron oxide nanoparticles exhibit unexpectedly beneficial magnetic properties (e.g., saturation magnetization) resulting from both the size of the nanoparticles and the iron (II) content. Accordingly, the iron oxide nanoparticles are attractive in general for a range of imaging, therapy and sensing applications (using alternating magnetic fields), and particularly for magnetic particle imaging. Methods of forming the iron oxide nanoparticles are also provided, such methods including a controlled oxidation step wherein a small amount (e.g., 1%) of gaseous oxygen is exposed to wüstite nanoparticles for a defined period of time sufficient to partially oxidize the wüstite but prevent conversion entirely to magnetite. Exposure of the iron oxide nanoparticles to 1% oxygen for longer periods of time result in the complete conversion to magnetite to produce magnetite nanoparticle with a shell of maghemite. Finally, methods of using the iron oxide nanoparticles are also provided. Representative methods include magnetic particle imaging (MPI), magnetic resonance imaging (MRI), and hyperthermia.

The nanoparticles are “under oxidized” compared to traditionally synthesized iron oxide nanoparticles. Specifically, the subject nanoparticles are in a metastable or intermediate state between wüstite (FeO) and magnetite (Fe₃O₄). This is accomplished by precisely controlling the oxidation of wüstite and terminating the oxidation prior to oxidation to magnetite.

As used herein, the term “about” indicates that the subject value can be modified by plus or minus 5% and still fall within the described and/or claimed embodiment.

Iron Oxide Nanoparticles

Iron oxide nanoparticles are provided that exhibit unexpectedly exceptional magnetic properties (e.g., relatively high differential susceptibility in AC magnetic field). The provided iron oxide nanoparticles achieve these properties at least in part to their unique composition. Particularly, the iron oxide nanoparticles include a core in a precisely tuned intermediate oxidation state in between wüstite and magnetite. This intermediate state is defined by the proportional amount of iron (II) to iron (III) within the cores of the nanoparticles, as will be described in further detail below. In short, the cores of the nanoparticles are oxidized to a certain extent from a starting material of wüstite (100% iron (III)) without fully oxidizing to magnetite (33.3% iron (II) and 66.6% iron (III)).

The iron oxide nanoparticles in the disclosed embodiments may include a number of distinct portions, including a core comprising an inner core and a shell, as well as a coating around the core. In certain embodiments the nanoparticles comprise an inner core, a shell, and a coating around the core (i.e. disposed adjacent the shell). In another embodiment, the nanoparticles comprise an inner core and a shell but no coating. In yet another embodiment, the nanoparticles comprise an inner core, no shell, and a coating disposed adjacent to the inner shell. As used herein, the term “core” refers to the iron oxide containing components of the nanoparticle. Every nanoparticle includes an inner core that is the center of the nanoparticle. The inner core excludes a shell. In embodiments that include a shell, the term “shell” refers to the oxidized shell at the surface of the iron oxide nanoparticle, which has typical thickness of 2 nm or less. The shell is iron oxide but is a different composition of iron oxide than the inner core. The inner core and the shell are monolithic and chemically bound, with the shell typically being a thin layer of the original wüstite iron oxide nanoparticle material that is oxidized. Unless stated otherwise, the shell composition is taken to be maghemite (Fe₂O₃) and 1.4 nm thick for determining the iron composition of the core.

In one aspect, a plurality of iron oxide nanoparticles is provided. In one embodiment, each iron oxide nanoparticle includes a core comprising an iron oxide inner core comprising an intermediate phase between stoichiometric FeO and stoichiometric Fe₃O₄, the inner core comprised of iron (II) in an amount of from 34% to 50% of the total iron in the inner core. As used herein, “stoichiometric FeO” refers to wüstite, wherein 100% of total iron is iron (II). As used herein, “stoichiometric Fe₃O₄” refers to magnetite, wherein 33.3% of total iron is iron (II) and 66.6% of total iron is iron (III).

Iron oxide nanoparticles are traditionally synthesized by fully oxidizing wüstite to magnetite. Until now there has been no method for producing under oxidized iron oxide nanoparticles and there has been no impetus to develop such a method. Magnetite is thought to be the superior form of iron oxide nanoparticles for imaging applications and there is no indication that under-oxidized iron oxide nanoparticles would have characteristics desirable for use in such imaging applications (e.g., MPI). However, the provided iron oxide nanoparticles demonstrate exceptional magnetic properties based on a combination of size and specific composition as defined by the iron(II) content.

When defining the amount of iron (II) in the inner core of a nanoparticle, it is important to note that if a shell exists around the core (e.g., a maghemite shell), the shell is assumed to have zero iron (II) and instead any iron is in the form of iron (III), such as in maghemite. Accordingly, if the inner core is comprised of iron (II) in an amount of from 34% to 50% of the total iron in the inner core, only iron (II) from the inner core factors into the calculation. The balance of iron in the inner core is oxidized iron (III). In one embodiment the iron (II)% in the inner core is 36% to 45% of the total iron in the inner core. In one embodiment the iron(II)% of the inner core is 39% to 43% of the total iron in the inner core.

Given the potential presence of a shell (e.g., maghemite) around the inner core of the iron oxide nanoparticle, a second aspect of the disclosure provides a plurality of iron oxide nanoparticles, each iron oxide nanoparticle comprising a core of iron oxide, wherein the plurality of iron oxide nanoparticles has an iron (II) content of 25-37% of the total iron content; and wüstite (FeO) is visible in a select area electron diffraction image obtained by transmission electron microscopy. In one embodiment, the iron (II) content of the core is between 27 and 35% of total iron. In one embodiment the iron (II) content of the core is between 28 and 34% of the total iron.

The second aspect is different than the first in that the nanoparticles are defined by total iron (II) content of the nanoparticles, as opposed to only the iron (II) content of the inner core (in the first aspect). In this second aspect, the total iron (II) content of the nanoparticle, including inner core and shell, if present, is 25-37%. The total iron (II) content can be determined directly by titration, as disclosed herein. This is in contrast to the iron (II) content of the inner core which must be calculated by assuming the presence of a 1.4 nm thick maghemite shell that contains no iron (II).

In the second aspect, the nanoparticles are further defined by wüstite (FeO) being visible in a select area electron diffraction image obtained by transmission electron microscopy. FIGS. 10A-10K and related text herein illustrate the wüstite character present in exemplary iron oxide nanoparticles.

In yet another aspect related to iron oxide nanoparticles, the nanoparticles are defined by both the first and second aspects:

Each iron oxide nanoparticle includes a core comprising an iron oxide inner core comprising an intermediate phase between stoichiometric FeO and stoichiometric Fe₃O₄, the inner core comprised of iron (II) in an amount of from 34% to 50% of the total iron in the inner core; wherein each iron oxide nanoparticle has a total iron (II) content of 25-37% of the total iron content; and wüstite (FeO) is visible in a select area electron diffraction image obtained by transmission electron microscopy.

The following embodiments are applicable to all of the aspects disclosed herein.

In one embodiment, the core further comprises a shell of iron oxide with a thickness of 0.7 nm to 2 nm surrounding the inner core. In a further embodiment, the shell is maghemite (Fe₂O₃). During any procedure in which a wüstite nanoparticle is oxidized a thin layer of maghemite will form, unless procedures are implemented to prevent the formation of such a maghemite shell. The thickness of the shell is on the order of one, or a few, unit cells. 0.7 nm to 2 nm estimates a typical maghemite shell thickness. 1.4 nm is considered to be an average maghemite shell thickness for the purposes of this disclosure.

In one embodiment, the iron oxide nanoparticles further comprise a coating layer disposed on an exterior surface of the core. The coating layer can serve several purposes, including acting as a surfactant or providing water-solubilizing properties to the nanoparticles.

As noted above, the core includes at least an inner core and in certain embodiments includes a shell. Accordingly, in one embodiment the coating layer is disposed directly on the inner core (no shell present). In an alternative embodiment, when a shell is present, the coating layer is disposed on the shell.

In one embodiment, the coating is attached to the core by a mechanism selected from the group consisting of covalent bonding, ionic bonding, van der Waals forces, and hydrophobic/hydrophobic interactions.

In one embodiment, the coating is a surfactant coating. A surfactant is used in certain methods of synthesizing representative iron oxide nanoparticles. As an example, oleic acid is a component of the thermolysis process used to form iron oxide nanoparticles in the methods and examples disclosed herein. In one embodiment, the surfactant coating comprises oleic acid, stearic acid, lauric acid, other fatty acids, oleylamine, or trioctylphosphine oxide.

In one embodiment, the coating is a water-solubilizing coating. A water-solubilizing coating is useful if aqueous storage or manipulation of iron oxide nanoparticles is desired. In one embodiment, the water solubilizing coating is a water-solubilizing agent or a water-solubilizing polymer. Exemplary water-solubilizing polymers include polysachharides (dextrans, etc.), polyethylenimine, polyethyleneglycol (PEG), polypropylene oxide, PMAO-PEG, R-PEG, where R is a group that binds to iron oxide, such as dopamine, silane, phosphine oxide, or other. Exemplary water-solubilizing “agents” include proteins, peptides, silica, aminopropyltriethoxysilane (APTES), etc.

In one embodiment, the coating comprises a surfactant coating disposed on the core and a water-solubilizing coating disposed on the surfactant coating. In this embodiment a dual coating is used to produce water soluble nanoparticles. This approach is described in the examples herein. Particularly, a surfactant (e.g., oleic acid) initially forms a surfactant coating on the nanoparticles, which renders them hydrophobic. Addition of a water-solubilizing coating on top of the surfactant coating provides water soluble nanoparticles.

In one embodiment, each nanoparticle is non-cubical in shape. In one embodiment, each nanoparticle is substantially spherical in shape. Nanoparticles formed using the exemplary methods disclosed herein are substantially spherical. However, due to the nanoscale dimensions of the nanoparticles, true spheres are difficult to form. Accordingly, “substantially” spherical nanoparticles may have facets and even take the form of a high-order polygon (e.g., dodecahedron).

Statistically, the plurality of nanoparticles is relatively monodisperse. The disclosed methods of synthesis provide uniform particle size and composition, which produces consistent properties, as needed for commercial use of the nanoparticles. In one embodiment, the plurality of iron oxide nanoparticles has a narrow distribution of diameters defined by a geometric standard deviation of 1.2 or less. In one embodiment, the plurality of iron oxide nanoparticles has a narrow distribution of diameters defined by a geometric standard deviation of 1.15 or less. In one embodiment, the plurality of iron oxide nanoparticles has a narrow distribution of diameters defined by a geometric standard deviation of 1.10 or less. Any number of nanoparticles can be synthesized, depending on the amount of starting materials used. However, in one embodiment, the plurality of iron oxide nanoparticles is 100 or more nanoparticles.

In one embodiment, the plurality of nanoparticles has a median diameter range of 10 nm to 40 nm. In one embodiment, the plurality of nanoparticles has a median diameter range of 20 nm to 35 nm. In one embodiment, the plurality of nanoparticles has a median diameter range of 23 nm to 30 nm. In one embodiment, the plurality of nanoparticles has a median diameter range of 24 nm to 28 nm.

Magnetic differential susceptibility, χ(H), of iron oxide nanoparticles is sensitive to their size and iron (II) content. In experiments presented in FIGS. 6A-9B, χ and Δ were mapped to Fe (II) composition and diameter and it was determined that certain combinations of Fe (II) composition and diameter are particularly suitable. As core diameter increases from 23 nm to 25 nm, and further to 28 nm, the preferred iron (II) content may increase, from about 26% Fe(II) in total, to about 29% Fe(II) in total, to about 30% in total, or about 36% Fe(II) in inner core, 40% Fe(II) in inner core, and 41% Fe(II) in inner core, respectively. A range of compositions is expected to provide excellent performance based on these results. Nanoparticles having exceptional magnetic performance include:

Accordingly, in one embodiment, the iron oxide nanoparticles have a median diameter of 20 nm to 40 nm and 34-50 Fe(II)% in the inner core or 25-37 Fe(II)% in total. In one embodiment, the iron oxide nanoparticles have a median diameter of 23 nm to 30 nm and 35-48 Fe(II)% in the inner core or 26-35 Fe(II)% in total. In one embodiment, the iron oxide nanoparticles have a median diameter of 24 nm to 28 nm and 36-46 Fe(II)% in the inner core or 27-33 Fe(II)% in total. In one embodiment, the iron oxide nanoparticles have a median diameter of 20 nm to 26 nm and 34-45 Fe(II)% in the inner core or 24-33 Fe(II)% in total. In one embodiment, the iron oxide nanoparticles have a median diameter of 27 nm to 40 nm and 40-50 Fe(II)% in the inner core or 28-37 Fe(II)% in total.

Mass magnetization (σ, magnetic moment per unit mass) is one metric by which to define the magnetic properties of the iron oxide nanoparticles. Mass magnetization can be measured according to techniques known to those of skill in the art (e.g., the saturation magnetic moment (A·m²), measured in a vibrating sample magnetometer (VSM), is divided by the iron mass (kg Fe), measured from inductively coupled plasma optical emission spectroscopy (ICP-OES), to give the mass magnetization (A·m²/kg Fe). The disclosed iron oxide nanoparticle demonstrates exceptional mass magnetization. In one embodiment, the plurality of iron oxide nanoparticles has a mass magnetization of 67 to 111 A·m²/kg Fe. In one embodiment, the plurality of iron oxide nanoparticles has a mass magnetization of 80 to 107 A·m²/kg Fe. In one embodiment, the plurality of iron oxide nanoparticles has a mass magnetization of 90 to 105 A·m²/kg Fe. In some applications, it is desirable to have large nanoparticles, for example 35-40 nm diameter. For such nanoparticles, it may be desirable to have mass magnetization that is lower than typical for similar size nanoparticles, to prevent aggregation of the nanoparticles in solution. Therefore, in one embodiment the disclosed plurality of iron oxide nanoparticles has low mass magnetization, of 67 to 105 A·m²/kg Fe. In other applications, it is desirable to have a high mass magnetization, for example to encourage aggregation for magnetic separation. Therefore in one embodiment the plurality of iron oxide nanoparticles has a high mass magnetization of 105-111 A·m²/kg Fe.

Saturation magnetization (Mₛ, when an increase in applied magnetic field will not increase the material magnetization further) is another metric by which to define the magnetic properties of the iron oxide nanoparticles. Saturation magnetization can be measured according to techniques known to those of skill in the art (e.g., the saturation magnetic moment (A·m2), measured in a VSM, is divided by the iron oxide nanoparticle volume (m³), determined from ICP and assuming a density of 5,180 kg/m³ for magnetite and 72 wt % Fe in magnetite, to give the saturation magnetization (A/m)). The disclosed iron oxide nanoparticle demonstrates exceptional saturation magnetization. In one embodiment, the plurality of iron oxide nanoparticles has a saturation magnetization of 250 to 415 kA/m. In one embodiment, the plurality of iron oxide nanoparticles has a saturation magnetization of 300 to 400 kA/m. In one embodiment, the plurality of iron oxide nanoparticles has a saturation magnetization of 330 to 390 kA/m. In one embodiment the plurality of iron oxide nanoparticles has low saturation magnetization of 250 to 390 kA/m. In one embodiment, the plurality of iron oxide nanoparticles has a high saturation magnetization of 390 to 415 kA/m.

Magnetic particle spectrometry (MPS) is a simple, common technique by which to measure magnetic properties of the iron oxide nanoparticles, including differential susceptibility, dm(H)/dH, or equivalently, χ(H). Again, the provided iron oxide nanoparticles demonstrate exceptional properties when measured by MPS. In one embodiment, the plurality of iron oxide nanoparticles has an intensity greater than 2.0×10⁻⁵ m³/gFe and full width at half maximum less than 6.5 mT/μ₀ as measured by a magnetic particle spectrometer at 25 kHz excitation frequency and 20 mT/μ₀ amplitude. In one embodiment, the plurality of iron oxide nanoparticles has an intensity greater than 2.5×10⁻⁵ m³/gFe and full width at half maximum less than 6.4 mT/μ₀ as measured by a magnetic particle spectrometer at 25 kHz excitation frequency and 20 mT/μ₀ amplitude. In one embodiment, the plurality of iron oxide nanoparticles has an intensity greater than 3.0×10⁻⁵ m³/gFe and full width at half maximum less than 6.3 mT/μ₀ as measured by a magnetic particle spectrometer at 25 kHz excitation frequency and 20 mT/μ₀ amplitude.

The iron oxide nanoparticles can be used for any application in which their properties would be beneficial. In particular the nanoparticles were designed with magnetic imaging and therapy applications in mind. Given the exceptional magnetic properties of the iron oxide nanoparticles, their use in any imaging and therapy applications known to those of skill in the art is contemplated. In one embodiment, the plurality of iron oxide nanoparticles is configured for use as magnetic particle imaging tracers.

In one embodiment, the plurality of iron oxide nanoparticles are magnetic tracers configured to be introduced into a subject. In this regard, the nanoparticles include a coating compatible with use in the subject. In one embodiment the subject is a mammal. In a further embodiment the subject is a human.

In one embodiment, the magnetic tracers are configured for use in a magnetic imaging technique selected from the group consisting of magnetic particle imaging and magnetic resonance imaging.

In one embodiment, the plurality of iron oxide nanoparticles are configured for use in a magnetic therapy selected from the group consisting of hyperthermia and sentinel lymph node biopsy.

Methods of Using Iron Oxide Nanoparticles

As noted above, the disclosed iron oxide nanoparticles have exceptional magnetic properties and can be used in any number of imaging and therapy techniques. Accordingly, in another aspect a method is provided that comprises applying a magnetic field to a plurality of iron oxide nanoparticles as disclosed herein.

Specific methods include magnetic imaging techniques such as magnetic particle imaging and magnetic resonance imaging. These techniques and the use of magnetic nanoparticles as agents are well known. The disclosed iron oxide nanoparticles can be used in any of the known methods. In one embodiment, the method is a magnetic particle imaging method and the magnetic field comprises a spatially varying magnetic field with a field-free region and a time varying magnetic field.

Magnetic hyperthermia is another specific technique that the iron oxide nanoparticles are compatible with. In one embodiment, the method is a magnetic hyperthermia method and the magnetic field is an alternating magnetic field configured to heat the plurality of nanoparticles.

Sentinel lymph node biopsy is yet another method in which the iron oxide nanoparticles are compatible with. In one embodiment, the method is a magnetic sentinel lymph node biopsy method, the method further comprising a step of detecting a magnetic response to the magnetic field.

Methods of Making Iron Oxide Nanoparticles

In one aspect, a method of forming a plurality of iron oxide nanoparticles is provided. In one embodiment, the method includes:

forming a plurality of iron oxide nanoparticles in a solution using thermolysis; and

oxidizing the plurality of iron oxide nanoparticles in the solution by exposure to a gas mixture comprising an inert gas and a gaseous oxygen (O₂) content of from 0.01 to 5% by volume to provide a plurality of oxidized iron oxide nanoparticles;

wherein the step of oxidizing the plurality of iron oxide nanoparticles takes place at an oxidation temperature of 200° C. to 370° C.

Generally, the synthetic methods can be used to oxidize iron to a higher oxidation state. While the oxidation of wüstite controllably towards magnetite is the emphasis of the disclosures herein, the methods are not limited to such embodiments, at the broadest level.

Importantly, the methods are safe, scalable, and reliable. Existing synthetic schemes for forming iron oxide nanoparticles fail in one or more of these categories. In particular, the use of thermolysis followed by exposure to air in order to generate (stoichiometric) magnetite iron oxide nanoparticles is unsafe when scaled to large volumes due to combustion dangers related to the organic solvents used when combined with oxidation with air (˜21% oxygen).

The methods can be used to form the iron oxide nanoparticles disclosed herein but can also be used to controllably oxidize non-spherical nanoparticles or to oxidize iron oxide between other states besides the wüstite to magnetite transition emphasized herein.

In particular, the methods are optimal for forming iron oxide nanoparticles useful in magnetic particle imaging (MPI) and other imaging and therapy applications requiring monodisperse and phase-controlled iron oxide nanoparticles.

The controlled oxidation methods disclosed herein not only improve safety and consistency of the synthesis of iron oxide nanoparticles, but also enable the formation of heretofore unknown compositions of iron oxide nanoparticles, specifically, the “under-oxidized” iron oxide nanoparticles disclosed in detail elsewhere herein. This is essentially because until now all iron oxide nanoparticle synthetic techniques sought to form magnetite and therefore oxidized wüstite as quickly as possible. It was unknown that intermediate oxidation states of iron oxide between wüstite and magnetite would exhibit the magnetic properties disclosed herein. In one embodiment, the plurality of oxidized iron oxide nanoparticle has an iron (II) content of 20 to 50% of the total iron content.

The methods can essentially adapt any known oxidation technique and replace it with the careful introduction of inert gas with a small amount of oxygen. Limiting the oxygen exposed to the nanoparticles in solution allows for the oxidation to be controlled with regard to the extent the nanoparticles are oxidized (i.e., controlling iron (II) content).

Importantly, the technique limits oxygen in solution in order to stay below the limiting oxygen concentration of all solvents within the solution. Particularly after a thermolysis reaction there may be organics of unknown composition in the solution that may have a low limiting oxygen concentration. If air or a greater amount of oxygen is used, the danger of combustion increases. Therefore, 5% oxygen is the maximum safe concentration of oxygen to use. Lower concentrations of oxygen increase safety or can be used to slow the oxidation reaction in order to control the composition of the iron oxide nanoparticles formed. The further lowering of the oxygen concentration would eventually result in slowing the oxidation rate as to make the oxidation time longer than is deemed practical. In one embodiment, the oxidation is performed with a gaseous oxygen (O₂) content of from 0.1 to 2% by volume. In one embodiment, the oxidation is performed with a gaseous oxygen (O₂) content of from 0.5 to 1.5% by volume.

In one embodiment, the step of oxidizing the plurality of iron oxide nanoparticles takes place with the introduced gas mixture containing a concentration of oxygen below the limiting oxygen concentration of any flammable liquids within the solution.

Temperature is an important aspect of the method, because the method allows for operation at relatively high temperatures without danger of combustion—a result of low oxygen in the system. As noted above, in the broadest embodiment, the step of oxidizing the plurality of iron oxide nanoparticles takes place at an oxidation temperature of 200° C. to 370° C. If air or greater oxygen concentration is used as the oxidant, the temperature must be reduced during oxidation below 200° C. for safety. 200° C. is not a limiting temperature of the disclosed methods. Instead, higher temperatures more consistent with typical thermolysis reactions can be used. In one embodiment, the oxidation temperature is 250° C. to 330° C. In one embodiment, the oxidation temperature is 300° C. to 325° C.

In one embodiment, the step of exposure to the gas mixture occurs of an exposure time period of 5 minutes to 72 hours. In one embodiment, the step of exposure to the gas mixture occurs of an exposure time period of 1 hour to 24 hours. The oxidation time can be highly dependent on the concentration (%) of oxygen in the gas mixture being introduced into the reaction, the flowrate of the gas mixture, the temperature of the reaction (nanoparticle solution) and the nature of the particles (e.g. diameter, iron oxide species).

In one embodiment, the gas mixture is introduced directly into the iron oxide nanoparticle solution. The introduction of the oxygen gas mixture into the reaction solution could be performed by a variety of methods known to those skilled in the art. Furthermore the rate of addition of the gas mixture into the reaction vessel or flowrate can be monitored and adjusted.

In one embodiment, the gas mixture is introduced into the reaction vessel above the nanoparticle solution. Furthermore the rate of addition of the gas mixture into the reaction vessel or flowrate can be monitored and adjusted. To further aid in the monitoring of the oxidation the concentration of oxygen in the atmosphere inside the reaction vessel can be monitored using an oxygen sensor.

In one embodiment, the step of oxidizing the plurality of iron oxide nanoparticles takes place with the introduced gas mixture containing a concentration of oxygen below the limiting oxygen concentration of any flammable liquids within the solution.

In one embodiment, the solution comprises a liquid selected from the group consisting of alkanes, alkenes, ethers, and/or amines, including octadecene, hexadecane, eicosene, docosane, octadecane, phenyl ether, dibenzyl ether, octyl ether, trioctylamine, and combinations thereof.

In one embodiment, the inert gas is argon or nitrogen.

In one embodiment, the thermolysis reaction comprises heating of an iron-containing complex selected from the group consisting of iron pentacarbonyl, iron tri(acetylactonate), iron oleate, iron stearate, and an iron carboxylate.

The nanoparticles formed in the method are of a shape defined by the starting nanoparticles prior to oxidization. In one embodiment, the plurality of oxidized iron oxide nanoparticles includes iron oxide nanoparticles according to the embodiments disclosed herein. In one embodiment, the oxidized iron oxide nanoparticles are spherical in shape. In one embodiment, the oxidized iron oxide nanoparticles have a median diameter of 10 nm to 40 nm.

While spherical nanoparticles are primarily discussed herein, it will be appreciated that the oxidation methods disclosed herein are not limited to oxidizing spherical nanoparticles. Any nanoparticle shape can be subjected to the oxidation methods. Accordingly, in one embodiment, the iron oxide nanoparticles have a shape selected from the group consisting of spheres, cubes, rods, polyhedrons, and plates.

### EXAMPLES

The following examples are included for the purpose of illustrating, not limiting, the disclosed embodiments.

### Example 1: Materials and Procedures for Synthesizing and Testing Iron Oxide Nanoparticles

The disclosed methods include a scalable process for producing iron oxide nanoparticles of uniform phase, and a composition of iron oxide nanoparticles rich in iron II, which provides unexpected and preferred magnetic behavior. The process includes first forming FeO nanoparticles in solution via a thermolysis reaction and subsequently oxidizing the FeO nanoparticles by adding a gas mixture containing approximately 1% oxygen in argon to the reactor until the desired iron oxide phase is achieved. The product nanoparticles can be iron oxide, with an inverse spinel crystal structure, containing 100% iron (III), or a mix of iron (III) and iron (II). The % of iron (II) can range from 100% to 0%.

Any samples referred to herein are in reference to Table 1, at the end of this section.

Oxidation to Fe₃O₄ with Oxygen in Inert Gas

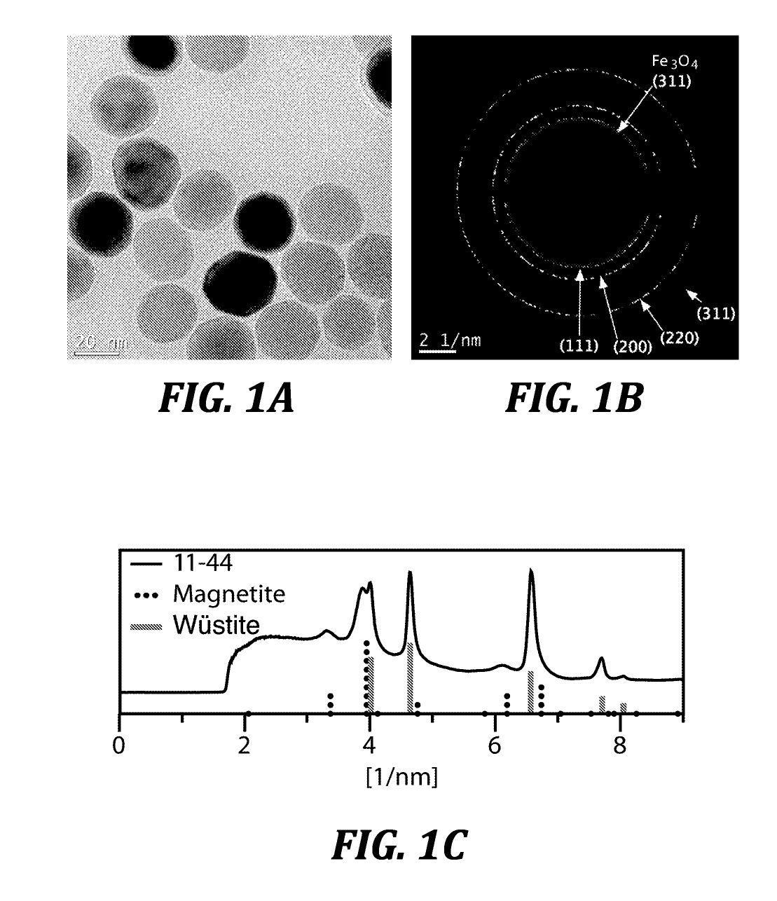

Thermolysis of iron oleate in oleic acid under an inert atmosphere favors formation of wüstite (FeO) nanoparticles. A “clean” batch of wüstite particles, with minimal oxygen exposure for example from inserting a needle for sampling, was prepared by stopping a 40 mmol Fe reaction after 2.5 hours at 324° C. and cooling under argon. High quality wüstite nanoparticles, 27.6 nm (0.08) diameter measured by TEM were obtained (FIGS. 1A-1C). FIG. 1A is a bright-field TEM image of the wüstite nanoparticles. A difference in contrast near the edges of the nanoparticles indicate the presence of a shell of lower density. Referring to FIG. 1B, the selected area electron diffraction (SAED) pattern (and radial integration, shown in FIG. 1C), indexing the diffraction rings shows the (111), (200), (220), and (311) lines characteristic of wüstite. In addition, several broad rings matching inverse spinel iron oxide were observed, which could come from the (311) and (220) of Magnetite or the (220) and (313) of Maghemite. In the FIGURES and examples where magnetite is identified by diffraction, it is generally understood that maghemite or intermediate phases could co-exist, unless otherwise specified. The spinel rings are broad and diffuse, in contrast to the sharper wüstite rings, suggesting small magnetite/maghemite crystallites due to oxidation of a thin shell on the particle surface, which could be formed during the sample preparation after air exposure.

Potassium permanganate titration showed these wüstite nanoparticles were 66% Fe(II). As seen in the bright field TEM image and the selected area electron diffraction in FIGS. 1A and 1B, the particles have a wüstite core and a thin shell (about 2 nm thick from bright field TEM) of inverse spinel iron oxide (Fe3O4 or γ-Fe2O3). If we assume a shell of pure γ-Fe2O3 (0% Fe(II)), since iron(II) readily oxidizes to iron (III) in air, total diameter of 27.6 nm and total Fe(II) of 66%, then a 1.7 nm thick shell is consistent with a pure wüstite core.

Interestingly, wüstite formation can go unnoticed during small-scale thermolysis common to many previous studies, (e.g. 2.5 mmol Fe, 100 ml reactor), since the small quantity of oxygen needed to transform the wüstite particles to inverse-spinel iron oxide can be supplied via minor leaks in the apparatus, such as dry ground-glass joints. However, a dedicated oxidation step is needed to achieve this transformation at larger scale and prepare magnetite nanoparticles with optimized magnetic moment. Other oxidants reported in the literature such as 3-chloroperbenzoic acid and trimethylamine N-oxide were considered too toxic and/or hazardous. Other groups have used air oxidation; however, introducing air to the thermolysis at 318° C. would be dangerous at large scale since octadecene auto-ignites at ˜250° C. Cooling the reaction for air-oxidation could be safer, and we determined that air oxidation of wüstite nanoparticles to magnetite nanoparticles proceeded at 175° C. and was rapid at 200° C., with the resulting particles having a clear electron diffraction pattern of magnetite. However, though below the auto-ignition temperature of octadecene, the addition of air to octadecene was still above the limiting oxygen concentration (LOC), making fire or explosions a concern for scalability.

To address these concerns, we developed an oxidation procedure using 1% oxygen in argon that is effective, simple, scalable, and can be performed safely at 318° C. by maintaining oxygen well below the estimated LOC (P. M. Osterberg, J. K. Niemeier, C. J. Welch, J. M. Hawkins, J. R. Martinelli, T. E. Johnson, T. W. Root, and S. S. Stahl, “Experimental Limiting Oxygen Concentrations for Nine Organic Solvents at Temperatures and Pressures Relevant to Aerobic Oxidations in the Pharmaceutical Industry,” Org. Process Res. Dev., p. 141223071236001, December 2014). Following this procedure, after wüstite nanoparticle formation had occurred, 1% oxygen in argon was bubbled into the thermolysis reaction mixture while maintaining the reaction temperature of 318° C. After optimizing the oxidation conditions for particle size, the resulting particles typically were highly magnetic, with saturation magnetization (Mₛ) of close to 400 kA/m, or 90% of bulk magnetite. Further analysis of the nanoparticles by x-ray and electron diffraction indicated they were composed of high purity magnetite. For a reaction using 40 mmol of Fe, typically, 1% oxygen was first added at ˜140 ml/min for 3 hours to perform most of the oxidation. To ensure the oxidation was complete without over-oxidizing the particles to maghemite, the flow of 1% oxygen was then reduced (15 mL/min) for an additional 28 hours (at 318° C.).

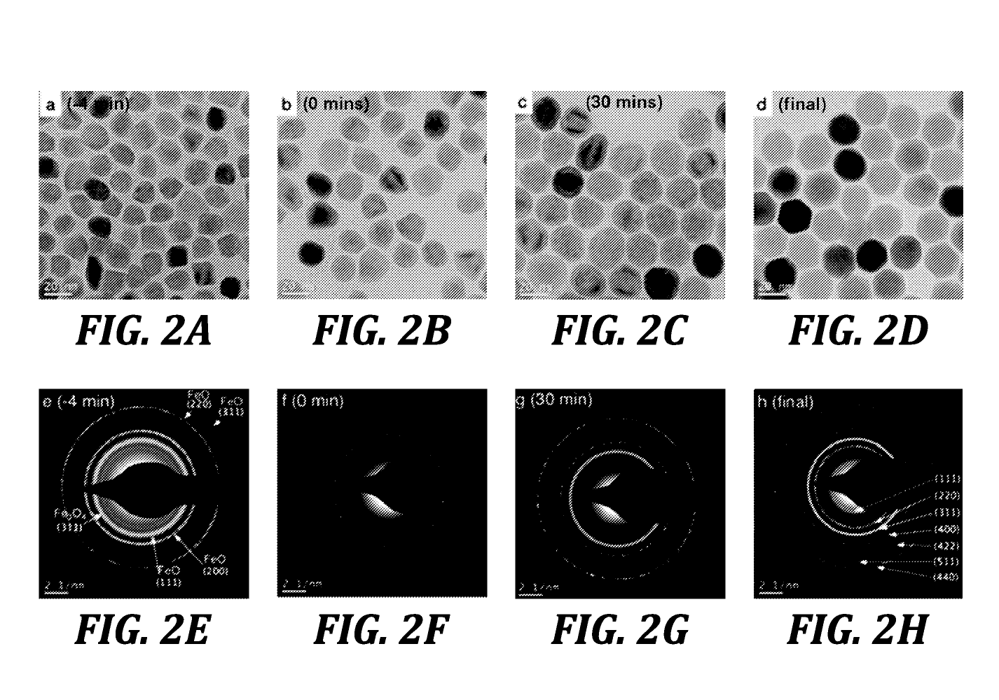

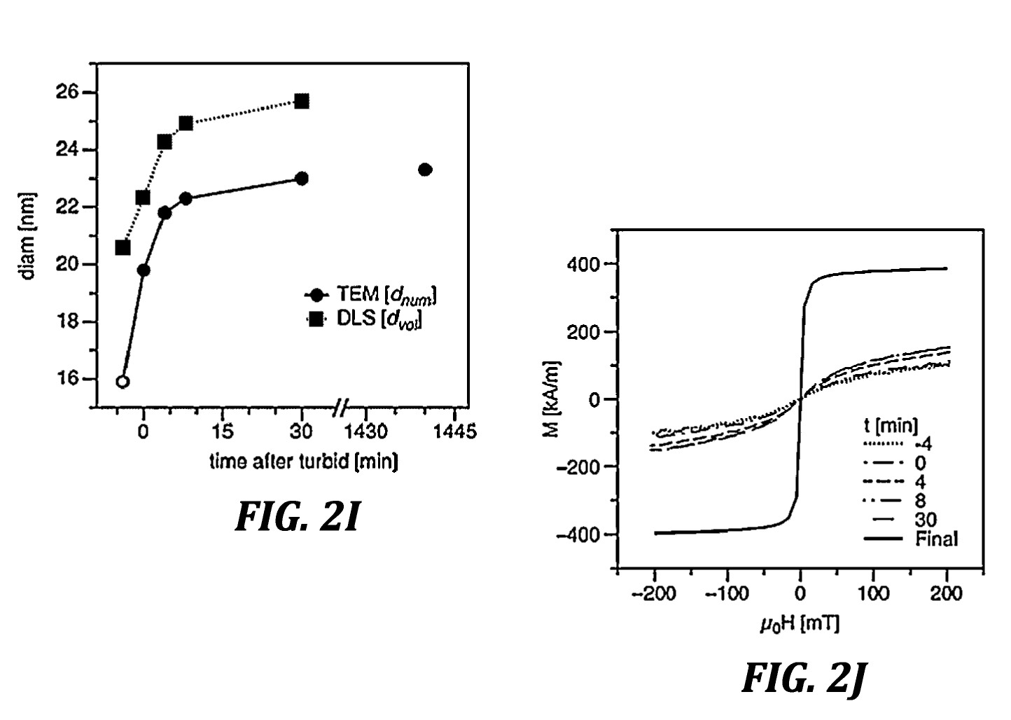

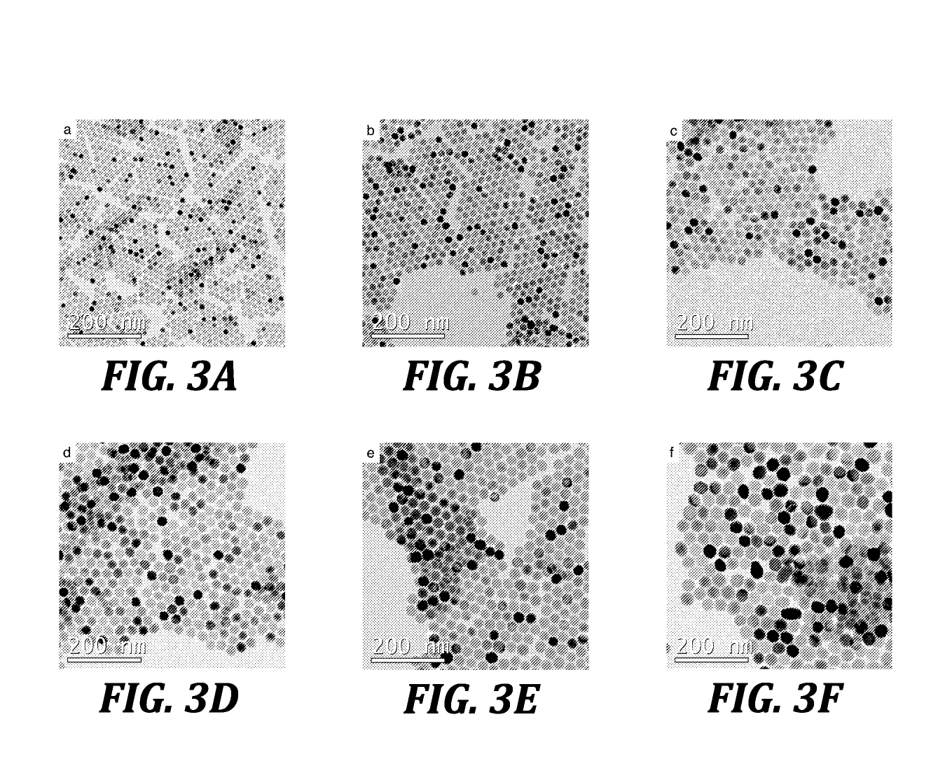

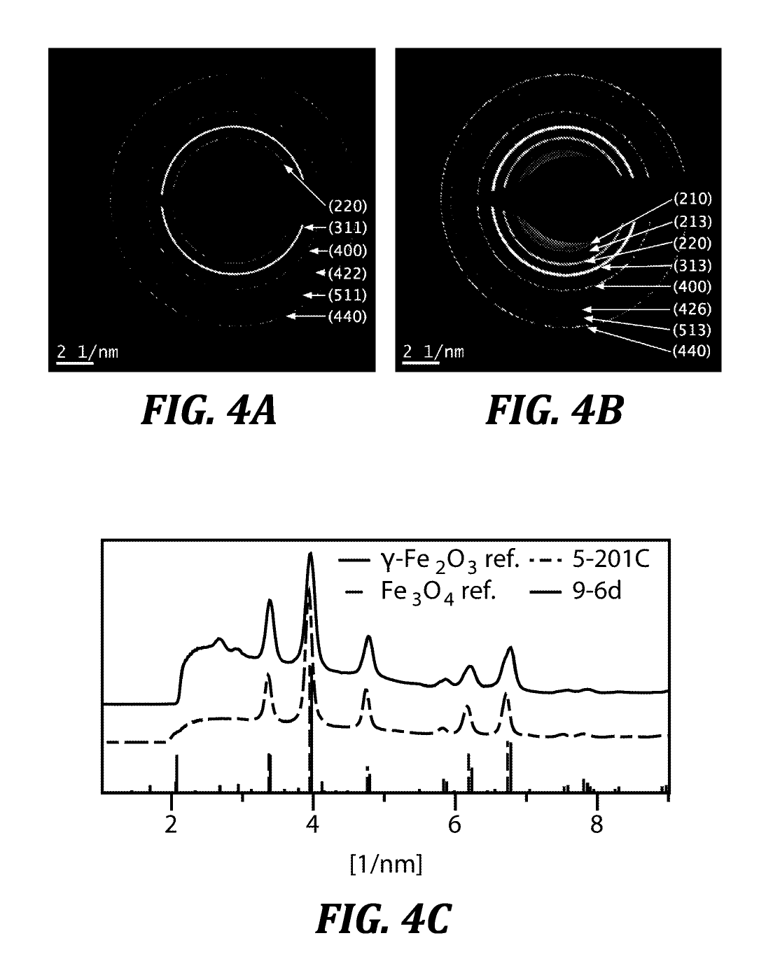

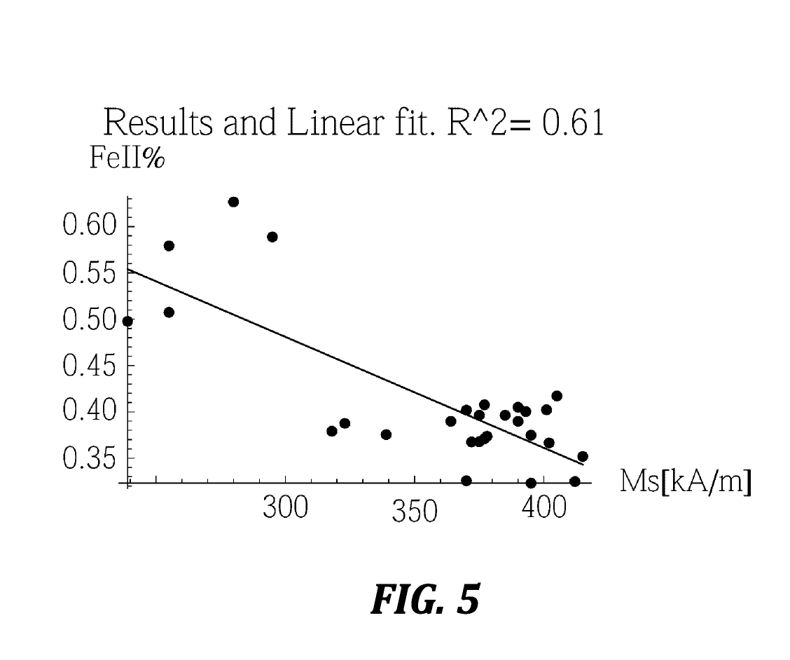

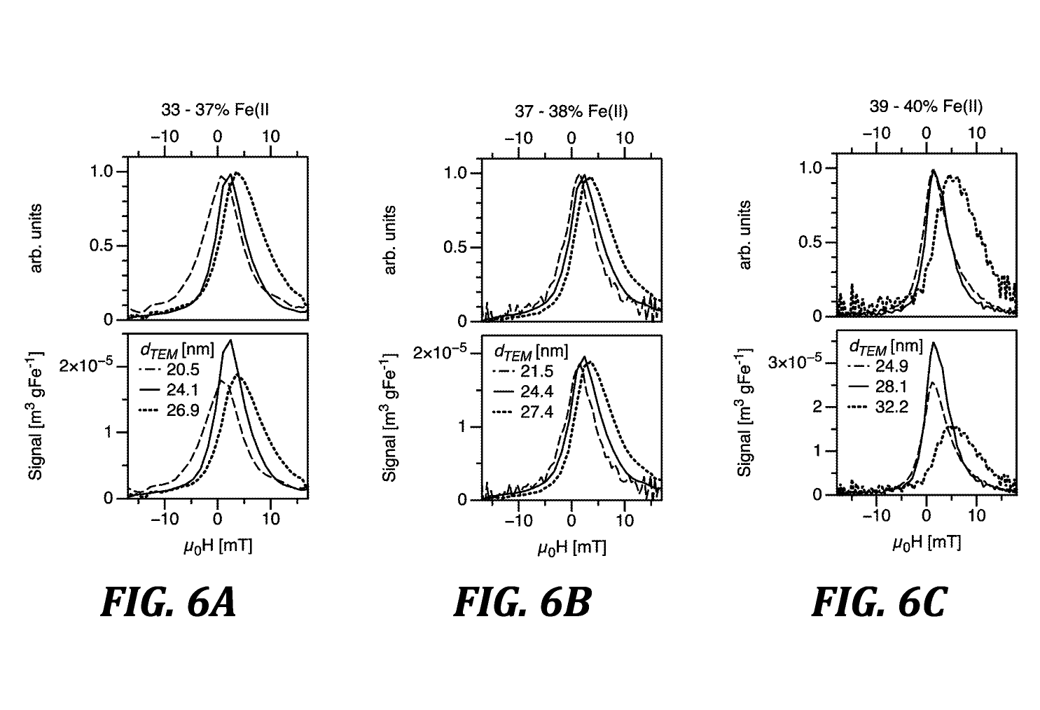

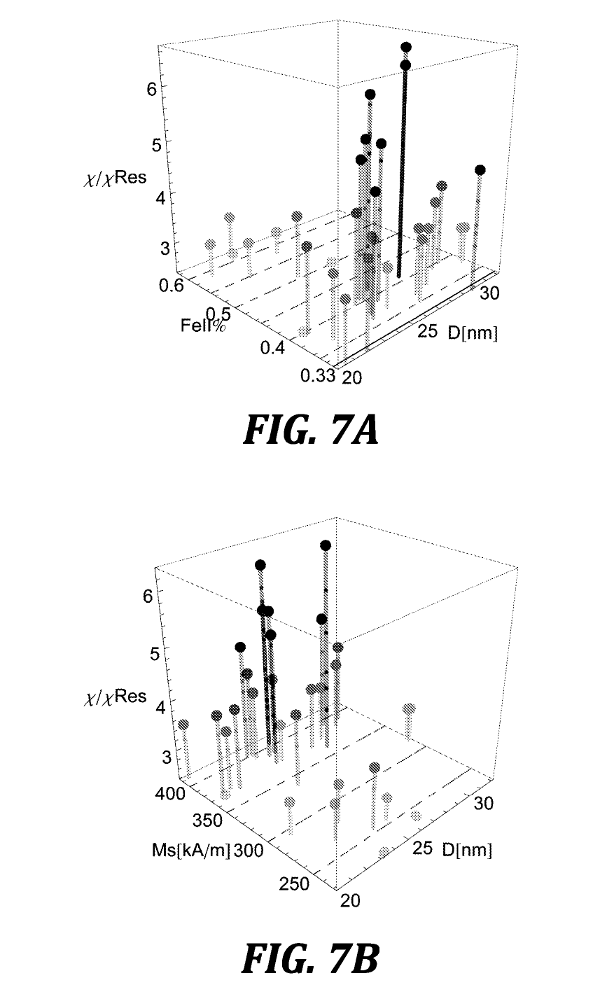

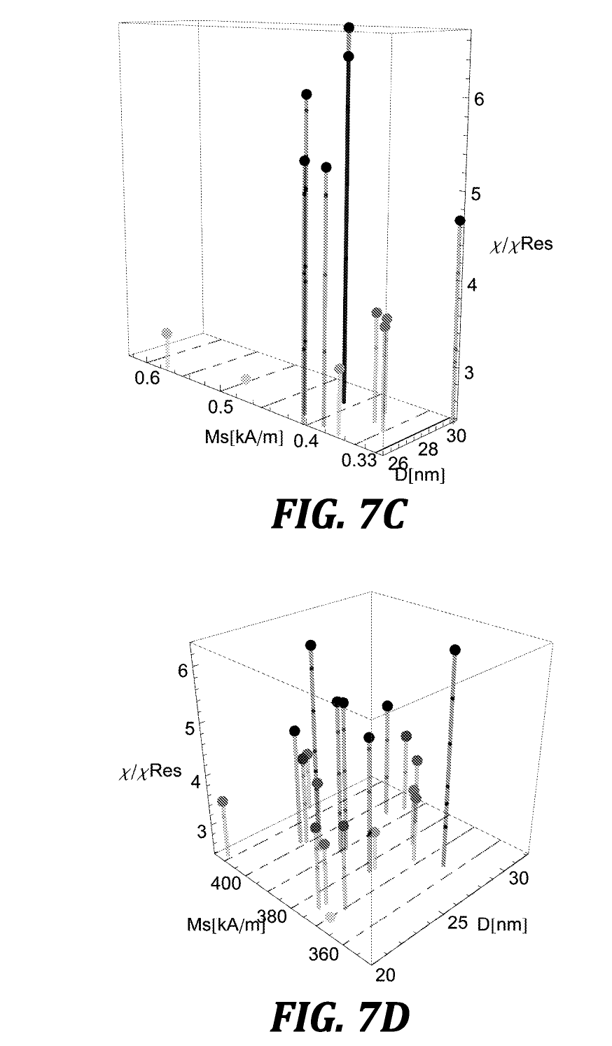

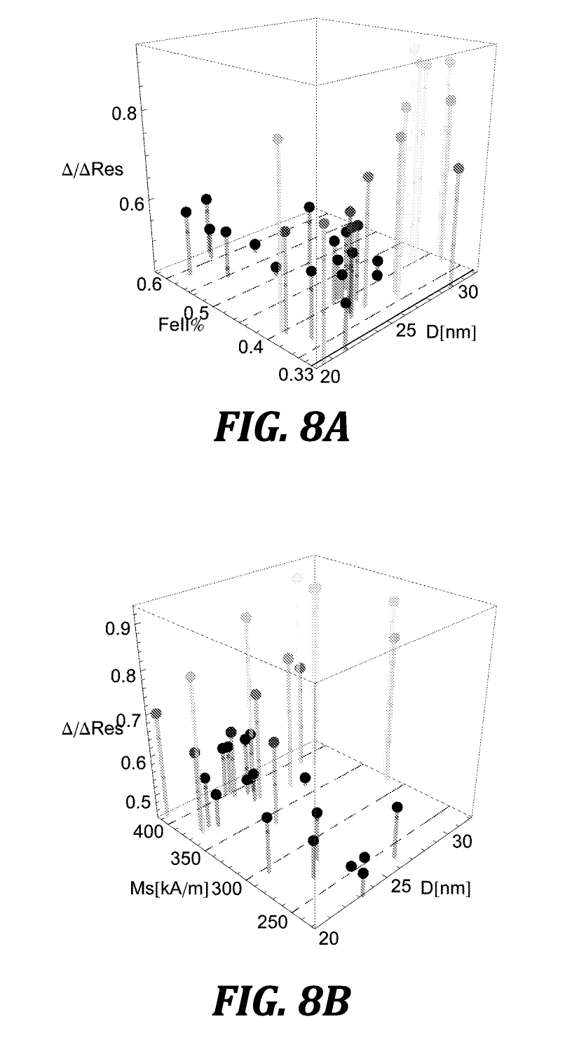

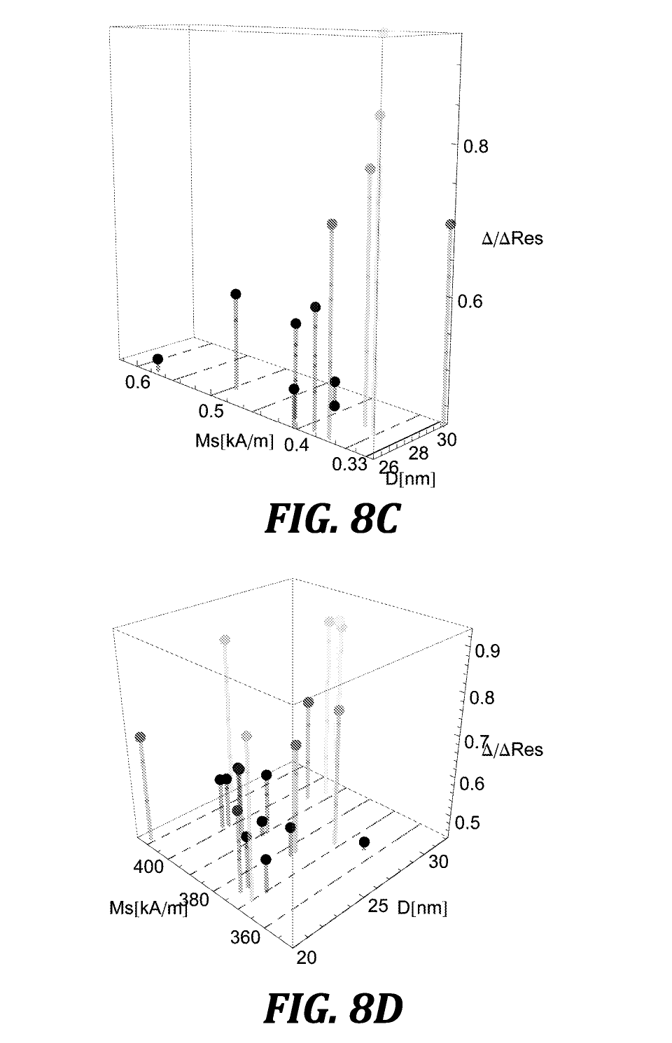

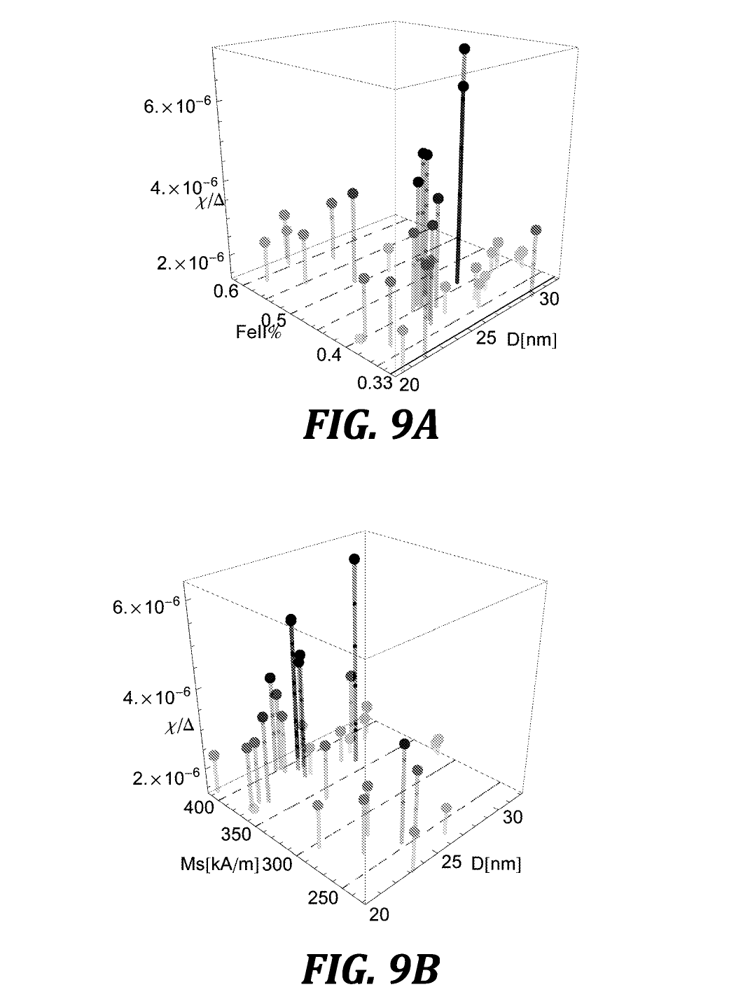

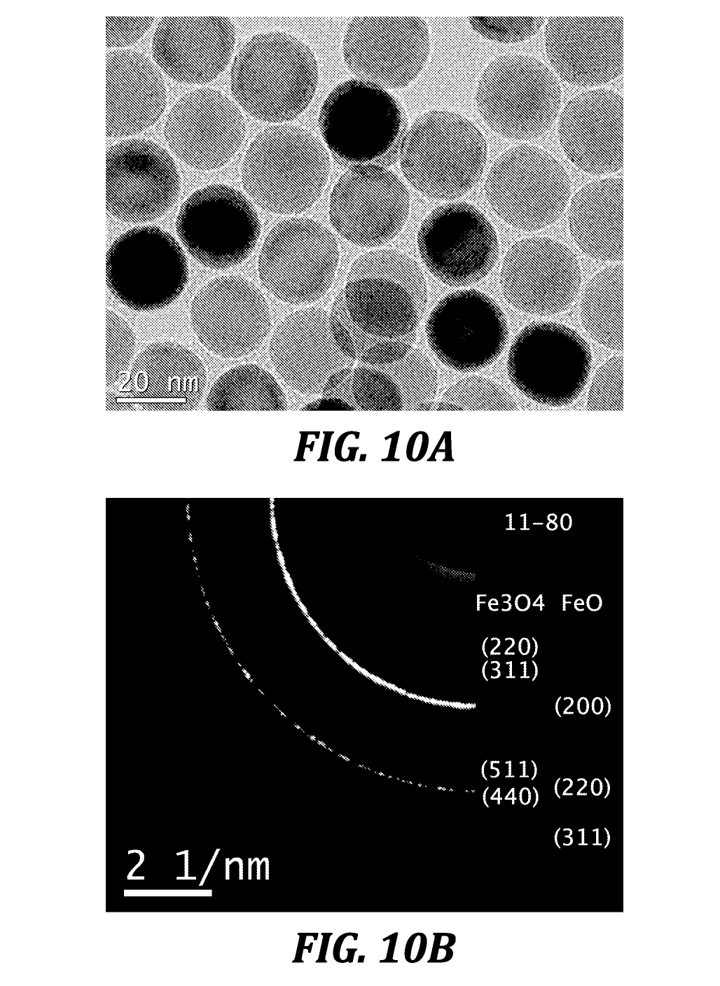

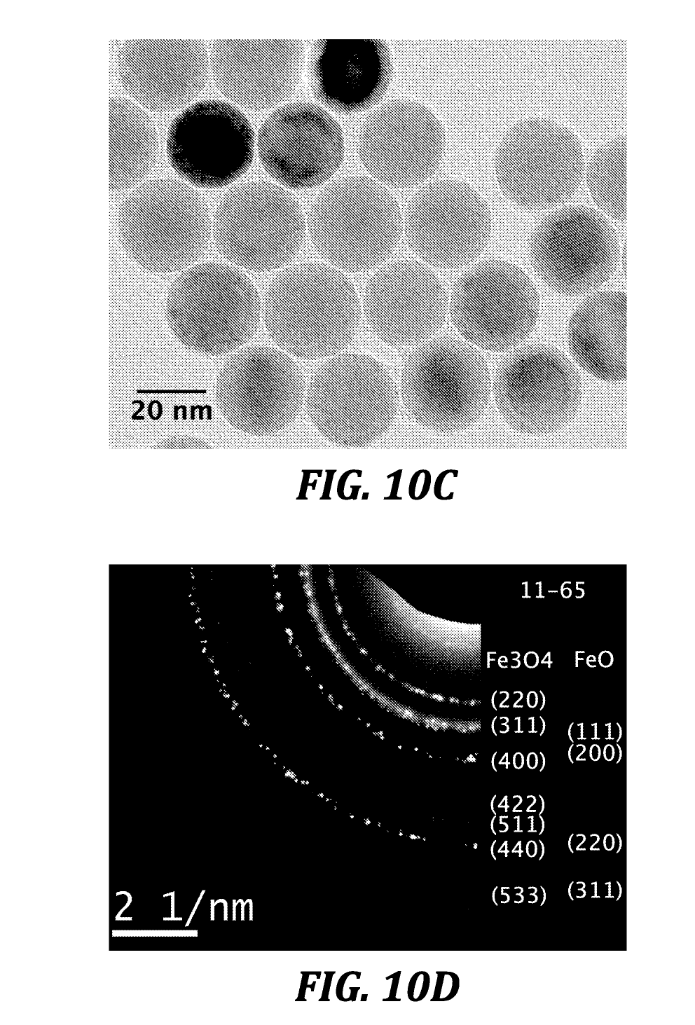

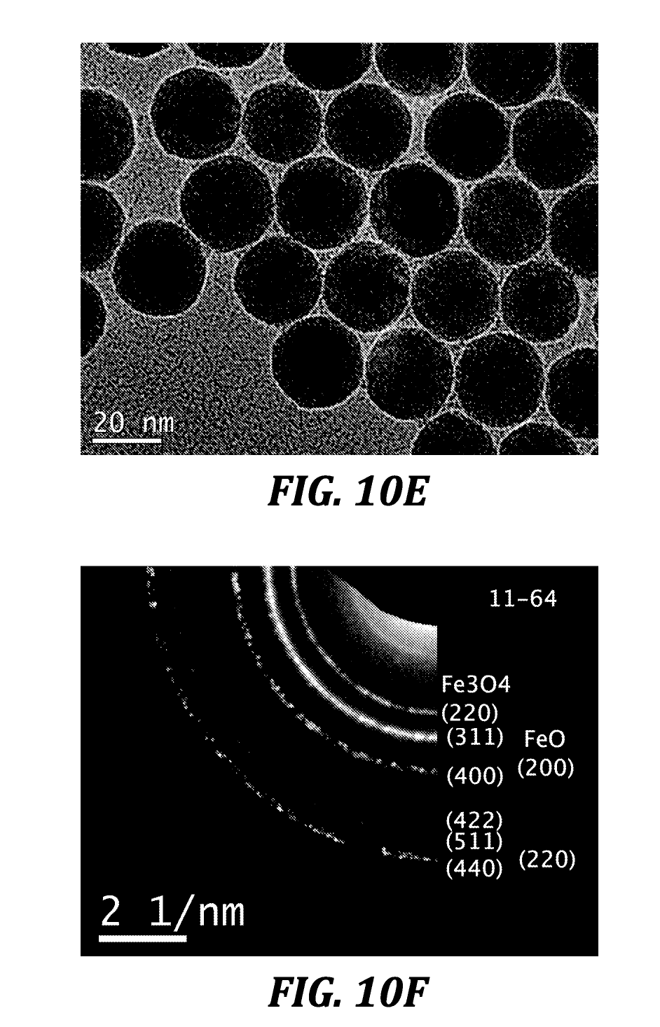

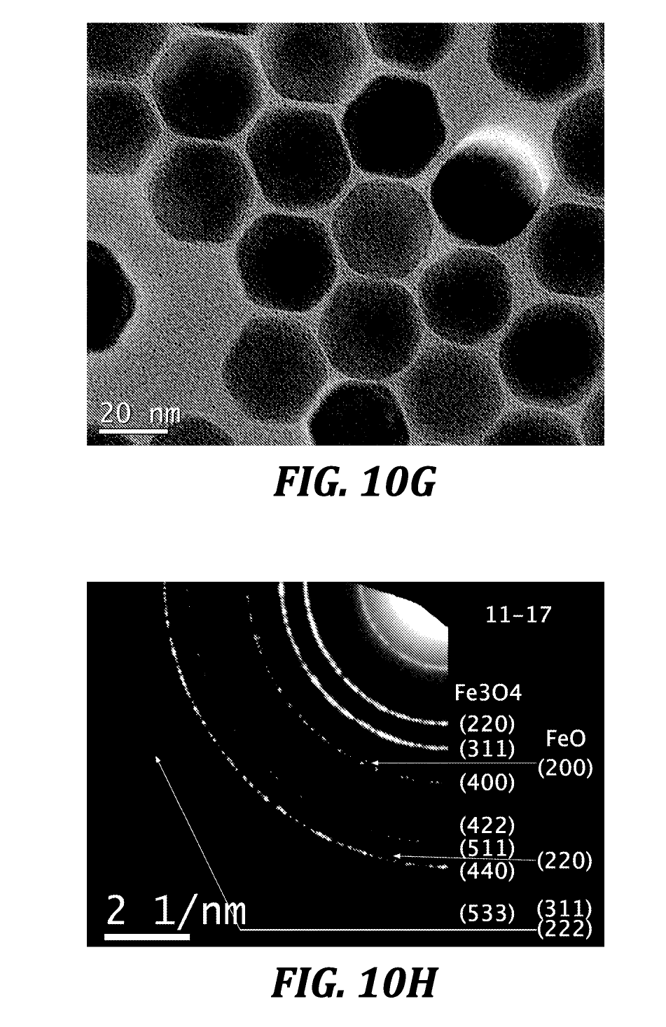

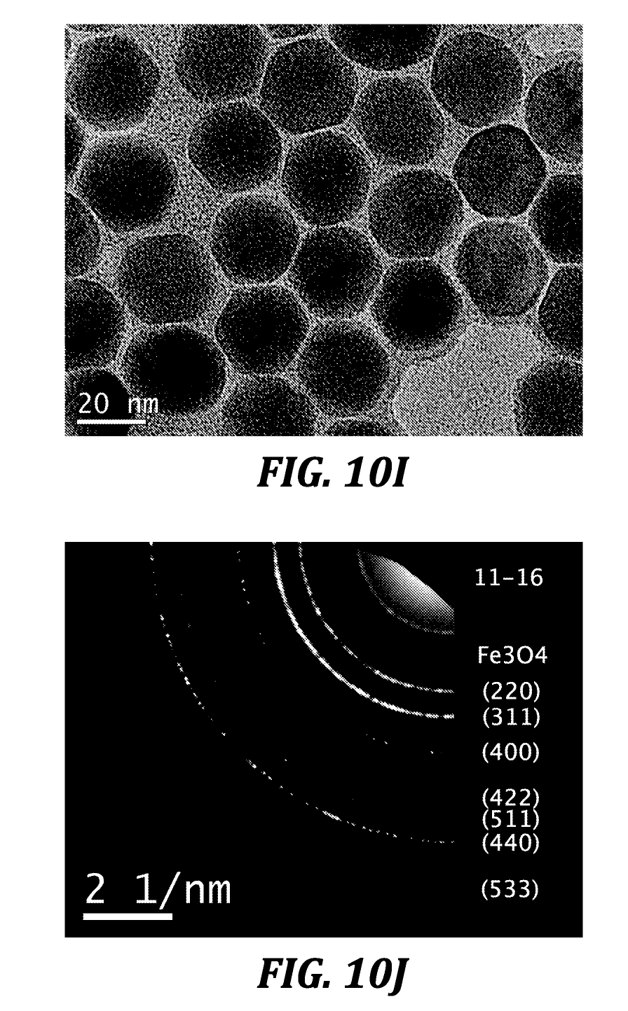

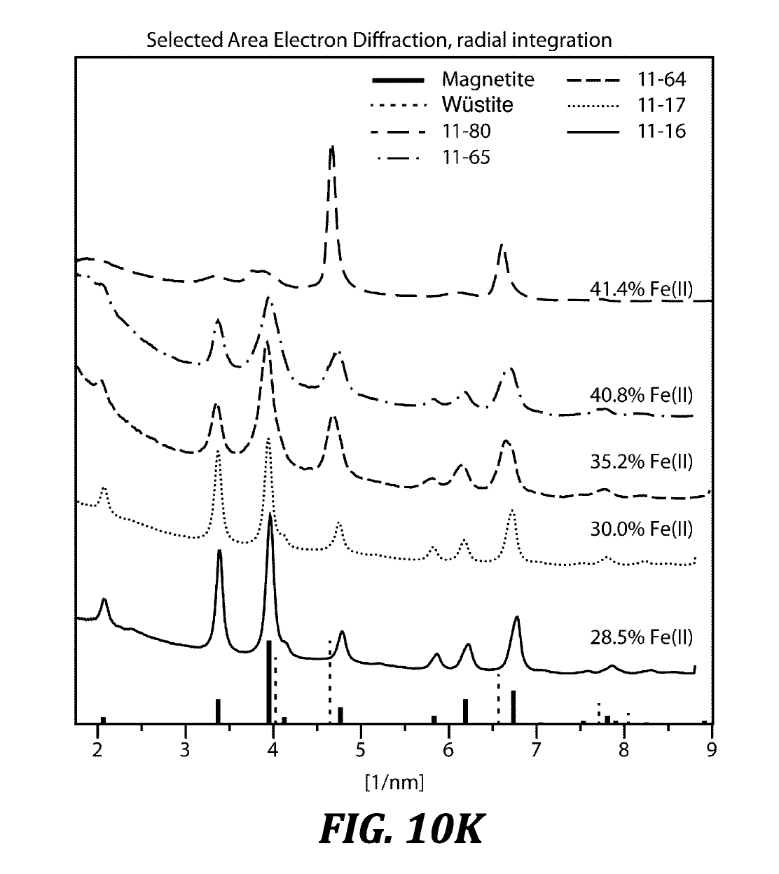

FIGS. 2A-2J illustrate nanocrystal evolution during the thermolysis reaction, including samples taken at early time points before the oxidation step, and a final point taken after oxidation with 1% oxygen in argon. Bright field TEM images of sample aliquots (FIGS. 2A-2D), with selected area electron diffraction are shown in FIGS. 2E-2H. Note that FIGS. 2D and 2H represent nanoparticles after the oxidation step, and the diffraction pattern in 2H is indexed to magnetite with (111), (220), (311), (400), (421), (511), and (440) lines clearly resolved. FIG. 21 graphically illustrates time evolution of particle size measured by TEM and DLS. DLS data is not shown for the final oxidized sample, since the strongly magnetic particles aggregated. FIG. 2J graphically illustrates magnetization curves measured at 295 K.

FIGS. 3A-3F. Bright field TEM images of nanoparticles with varying diameter made using the 1% oxygen in argon oxidation process. FIG. 3A: 15 nm, FIG. 3B: 20 nm FIG. 3C: 24 nm, FIG. 3D: 27 nm, FIG. 3E 30 nm, and FIG. 3F 35 nm.

FIGS. 10A-10J are bright-field TEM images and selected area electron diffraction images of examples of iron oxide nanoparticles that were titrated to determine their iron (II) %; and FIG. 10K illustrates radial integrations for each of the diffraction patterns. Sample numbers can be matched to those in Table 1 at the end of this section. The diffraction rings in FIGS. 10 B, 10D, 10F, 10H, and 10J were indexed to identify the phases of iron oxide present in the nanoparticles. The examples include core iron (II)% ranging from 28.5 to 41.4%, and reported values were not adjusted to account for an oxidized shell. The nanoparticles were formed using the controlled oxidation methods disclosed herein. The data presented illustrates the controllable iron (II) content achievable through controlled oxidation of a starting wüstite nanoparticle. In particular, FIG. 10K graphically illustrates the gradual compositional shift from a wüstite character in the highest Fe(II) samples to stronger magnetite character in the lower Fe(II) samples. Controlled oxidation therefore provides a powerful tool with which to precisely produce a desired mix of iron (II) and iron (III) in an iron nanoparticle.

An exemplary iron oxide nanoparticle with 41.4% iron (II) is presented in FIGS. 10A and 10B. Referring to FIG. 10B, both wüstite and magnetite phases are visible. The wüstite rings are intense and sharp compared to the broad, low-intensity lines for magnetite, suggesting these nanoparticles contained predominately wüstite, with a shell of magnetite.

An exemplary iron oxide nanoparticle with 40.8% iron (II) is analyzed in FIGS. 10C and 10D, and in FIG. 10D both wüstite and magnetite phases were observed. In this example, the wüstite and magnetite lines are similar in intensity.

An exemplary iron oxide nanoparticle with 35.2% iron (II) is analyzed in FIGS. 10E and 10F. The diffraction pattern (FIG. 10F) reveals wüstite and magnetite phases.

In example nanoparticles presented in FIGS. 10G and 10H, the titrated iron (II) % was 30.0%. In this example, the diffraction pattern is similar to pure magnetite, but with several additional spots due to wüstite (200), (220), and (difficult to see due to lower intensity) (222).

Finally, in FIGS. 10I and 10J is an example with 28.5% iron (II). This diffraction pattern (FIG. 10J) is standard magnetite.

Radial integrations for each of the diffraction patterns are presented in FIG. 10K, along with standards for magnetite and wüstite. After integration, what appear to be two distinct, neighboring diffraction lines in the diffraction images (e.g. FIGS. 10B, 10D, 10F, et al.), for example Fe₃O₄ (440) and FeO (220), appear as a single broad line.

Determination of Fe(II) Content in Nanoparticles by Titration.

Monitoring the oxidation was important, because the target phase Fe₃O₄ exists on a continuum containing different mixtures of iron II and iron III. Diffraction provides qualitative phase assessment for iron oxides, but is not perfect. Intermediate phases also occupy the inverse spinel structure and their primary diffraction peaks are similar. Some variations in minor peaks are expected, but often can't be distinguished for crystals with the desired size of 20-30 nm. Therefore, in addition to diffraction, we implemented potassium permanganate titration as a simple and quantitative method to monitor the iron II content during oxidation and determine precisely how much oxygen was required to yield the desired phase. Iron oxide nanoparticles dissolve rapidly in concentrated HCl, making it the ideal acid for digestion. Initially we had avoided the use of hydrochloric acid due to the common belief of interference by chloride with the permanganate titration. Further investigation suggested titration of Fe(II) samples in 0.5 M HCl solutions was possible, leading us to digest nanoparticles in concentrated HCl, then dilute the mixture by 20 to 1 with water, followed by permanganate titration. The end point for the titration is not the typical appearance of pink, but instead a sharp change from pale yellow to pale orange.

The estimated error in the titration measurement is about 1% Fe(II).

Effect of Particle Size and Solvent on Oxidation Rate

We also considered the influence of particle size and solvent on oxidation rate. As anticipated the size of the nanoparticles affected the oxidation rate. On the scale of 40 mmol Fe, only 6.7 mmol oxygen is theoretically required to transform wüstite to magnetite. In practice, small (20.5 nm) particles required about 24 mmol of oxygen to obtain pure magnetite, whereas larger particles (26.9 nm) required about 64 mmol of oxygen. The excess oxygen is either not absorbed or is consumed oxidizing the 1-octadecene.

1-octadecene plays a small role in reducing iron II during thermolysis. Unsurprisingly, octadecene is also oxidized along with nanoparticles during the oxidation step. We demonstrated the solvent influence by dispersing nanoparticles in octadecene and octadecane and exposing both to air at 175° C. for 30 minutes, with selected area diffraction data of both samples illustrated in FIGS. 4A-4C. FIG. 4A shows the diffraction pattern typical of samples oxidized using octadecene as solvent, whether air oxidation or 1% oxygen in argon, while in FIG. 4B, the maghemite (210) and (213) are visible, which was found only when using octadecane as solvent. The octadecene reaction remained black; however the octadecane reaction turned orange-red in color and lost all traces of black. In addition to color variations, we characterized the particles with electron diffraction. While the diffraction patterns of maghemite and magnetite substantially overlap, some fine features can be resolved if the crystal size is sufficiently large. Selected area electron diffraction of these samples (FIGS. 4A and 4B) show slight variations, including the (210) and (213) maghemite rings, which are visible only in the octadecane-oxidized sample. This result suggests octadecene is also being oxidized during the oxidation step, preventing conversion of magnetite to maghemite. This effect could serve as an important oxidative buffer, preventing over oxidation of iron oxide nanoparticles.

Compositions of Iron Oxide Nanoparticles with Iron II-Rich Cores

By varying the duration and flow rate of 1% oxygen in argon mixture, the oxidation procedure described above can be used to vary the amount of Fe(II) and Fe(III) in iron oxide nanoparticles within a range of about 66% Fe(II) to about 20% Fe(II). The Fe(II) composition can be determined using the titration procedure described herein. Consequently, the phase of the product iron oxide nanoparticles can be FeO, Fe₃O₄, Fe₂O₃, or a mixture containing one or more of these phases, or stable intermediates with non-stoichiometric quantities of Fe(II) and Fe(III). Results are provided in Table 2 at the end of this section.

Core/Shell Structure: Fe(II)-Rich Core with Fe(II)-Poor Shell

It is known from the literature that iron and iron oxide nanoparticles typically have a maghemite shell with thickness of up to a few nanometers, since the nanoparticle surface is exposed to oxygen which rapidly oxidizes iron II to iron III. For example, in J. Santoyo Salazar, L. Perez, O. de Abril, L. Truong Phuoc, D. Ihiawakrim, M. Vazquez, J.-M. Greneche, S. Begin-Colin, and G. Pourroy, “Magnetic Iron Oxide Nanoparticles in 10-40 nm Range: Composition in Terms of Magnetite/Maghemite Ratio and Effect on the Magnetic Properties,” Chemistry of Materials, vol. 23, no. 6, pp. 1379-1386, March 2011, Mossbauer and IR spectroscopy were used to characterize a maghemite (Fe(III) only) shell on magnetite nanoparticles with diameter 10-40 nm. The shell thickness varied with particle diameter, and was between 1.4 and 1.8 nm for particles between 25 and 30 nm diameter. In R. Frison, G. Cernuto, A. Cervellino, O. Zaharko, G. M. Colonna, A. Guagliardi, and N. Masciocchi, “Magnetite-Maghemite Nanoparticles in the 5-15 nm Range: Correlating the Core-Shell Composition and the Surface Structure to the Magnetic Properties. A Total Scattering Study,” Chemistry of materials, vol. 25, no. 23, pp. 4820-4827, 2013, magnetite particles ranging from 5 to 15 nm diameter were analyzed, and maghemite shell thicknesses were determined by x-ray scattering during synchrotron measurements. The maghemite shell thickness varied from 1.1 to 3 nm in that study.

Unless stated otherwise, the shell composition is taken to be maghemite (Fe₂O₃) and 1.4 nm thick for determining the iron composition of the core. Measurement of the exact shell thickness is difficult to perform and so 1.4 nm is used as the assumed shell thickness, based on accepted values presented in the prior art related to fully oxidized magnetite iron oxide nanoparticles and the following titrations of significant examples.

We used titrated iron (II) composition to determine maghemite shell thickness for the inventive nanoparticles using examples that were highly oxidized and minimally oxidized The maximum iron (II) composition was estimated by producing a sample (of mean diameter 27.6 nm) and omitting the oxidation step. In this example, the titrated Fe(II) percent was 66%, and an oxidized shell was evident on the surface of the nanoparticles in electron micrographs (see FIG. 1A), even though no oxidation step was included. Electron diffraction revealed the nanoparticles to contain wüstite (FeO) and inverse spinel iron oxide, either maghemite or magnetite, which have indistinguishable diffraction patterns for small crystals such as in the shell, with thickness estimated from bright field TEM images to be 2-4 nm. By assuming a core/shell model with a shell of pure maghemite (0% Fe(II)) the iron composition of the core can be determined. For 66% total iron II, the core composition varies form 91% to 100% as the shell thickness varies from 1.4 to 1.8 nm (the range of shell thicknesses expected for a 25 nm diameter nanoparticle reported in J. Santoyo Salazar, L. Perez, O. de Abril, L. Truong Phuoc, D. Ihiawakrim, M. Vazquez, J.-M. Greneche, S. Begin-Colin, and G. Pourroy, “Magnetic Iron Oxide Nanoparticles in 10-40 nm Range: Composition in Terms of Magnetite/Maghemite Ratio and Effect on the Magnetic Properties,” Chemistry of materials, vol. 23, no. 6, pp. 1379-1386, March 2011), indicating the core was relatively pure FeO.

In another example, wüstite nanoparticles were oxidized completely by flowing 1% O₂ in Ar at 60 mL/min for 28 hrs to ensure complete transition to the spinel phase magnetite or maghemite. Titration of a 26.9 nm diameter sample (9-145) revealed a 25.3% Fe(II). Since nanoparticles were fully oxidized to ensure the absence of wüstite, we can use the core/shell model to assume a shell of pure maghemite (0% Fe(II)), and that the measured 25.3% Fe(II) resides entirely in a magnetite core. This results in a shell thickness of 1.1 nm. Thus, our measured Fe(II) titration values of un-oxidized and extensively oxidized nanoparticles gives us an average maghemite shell thickness of 1.4 nm. Unexpectedly, we discovered that compositions of iron oxide nanoparticles with certain mixtures of Fe(II) and Fe(III), as determined by potassium permanganate titration, and supported by measurement of saturation magnetization, showed surprisingly improved magnetic characteristics for applications such as Magnetic Particle Imaging. In particular, the rate of change of magnetization in response to an alternating magnetic field was substantially increased when the nanoparticles contained between 34% and 50% Fe(II).

The desirable nanoparticles also feature saturation magnetization between 340 and 390 kA/m, which is less than typical for magnetite (˜446 kA/m for bulk Fe3O4). Reduced saturation magnetization is intrinsically linked to the composition and may play a role in causing improved AC magnetization. Furthermore, the nanoparticles tend to aggregate less than fully oxidized nanoparticles of the same size, making the under-oxidized nanoparticles are easier to process in solution. This behavior may be due to reduced magnetic moment, which decreases magnetic interaction between particles. The relationship between measured Fe(II)% and magnetic moment, represented by saturation magnetization, Mₛ, is illustrated in FIG. 5. Fe(II)% is displayed based on a 1.4 nm shell of maghemite. It is evident that magnetic moment decreases with increasing Fe(II)%, as would be expected, with the range of Ms values falling between 250 and 415 kA/m.

Particle Sizes.

Nanoparticles ranging in size from 20-32 nm were synthesized with under-oxidized iron composition.

Evolution of Magnetic Properties During Oxidation

Increase of magnetic moment is one feature of the compositional evolution during oxidation from wüstite to Magnetite. Another is evolution of the dynamic magnetic response χ(H), to a time varying field, H, which determines a nanoparticle's suitability for MPI and other applications based on AC magnetic excitation and dynamic magnetization. χ(H) can be characterized by the maximum intensity (which we represent using χ), and the full width at half maximum (FWHM), which we represent using Δ. Overall a greater χ is preferred, and smaller Δ is also preferred.

In this section, several presentations of data (FIGS. 6A-9B) are provided to illustrate the range of desirable compositions claimed and some identifying physical properties. A set of samples with a range of compositions was analyzed to determine Fe(II)% in the core, Mₛ, size and size distribution. To evaluate suitability for MPI and other applications, dynamic magnetization properties were measured by an MPS, an AC magnetometer that simultaneously applies an AC magnetic field at 25 kHz and 20 mT/μ₀, and measures χ(H). FIGS. 6A-6C provides complete representations of χ(H) for several samples of different Fe(II)) composition. Large χ and small Δ are desirable, preferably both in the same sample. In FIGS. 6A-6C, the AC response evolution is shown for increasing Fe(II)%. Δ is initially narrow at low Fe(II)%, and remains that way during early evolution while the signal intensity (χ) increases within the desired composition range. As oxidation proceeds, Δ begins to increase, and the peak position shifts to higher fields, indicating increased anisotropy in the nanoparticles or greater energy required to reverse the magnetization. As a result, the signal intensity decreases. A key finding here is that for a given Fe(II) composition, there is an optimal size.

To illustrate the interdependence of nanoparticle diameter and composition, results for a larger set of samples were plotted in FIGS. 7A-9B. For these FIGURES, χ and Δ were normalized using the measurements for Resovist (χ_{Res}=5.2×10⁻⁶ m³/gFe and Δ_{Res}=11.3 mT/μ₀), a commercial SPIO. Particles with 28 nm diameter and cores containing about 40% Fe(II) showed excellent performance. In some cases, individual samples may show good performance in one metric, but not another. For example, many samples with Fe(II)% greater than 0.45 showed good Δ but poor χ. Large size (>30 nm) and low Fe(II) (<34%) (also high Mₛ) resulted in poor performance and the greatest Δ. The physical origin of this phenomena may be due to larger coercive field, which requires more energy to reverse magnetic moment.

FIGS. 6A-6C: Evolution of χ(H) with oxidation for nanoparticles of different size, measured at 25 kHz applied field, 20 mT/μ₀ amplitude. FIG. 6A shows results for particles with cores between 33 and 37% Fe(II); FIG. 6B shows results for cores with 37-38% Fe(II); and FIG. 6C shows inner cores with 39-40% Fe(II). Top portions of FIGS. 6A-6C are normalized for comparing the peak width, (Δ), while bottom portions of FIGS. 6A-6C show intensity per unit iron.

FIGS. 7A-7D: Summary results of intensity of χ(H) for a range of sample compositions, presented with Fe(II)% (FIGS. 7A and 7B) and Ms (FIGS. 7C and 7D) used to characterize the composition. Results were normalized by χRes, the value measured for Resovist. Large values of χ are preferred. FIG. 7B shows a slice of the full set with narrow range of diameters, indicating the trend apparent.

FIG. 8A-8D: Results for Δ (FWHM of χ (H), (at 25 kHz, 20 mT/μ₀). Each bar represents a unique sample. Here, smaller Δ is preferred, as it represents better performance. Results were normalized by ΔRes, the value measured for Resovist. FIGS. 8A and 8B show Δ vs size, and Fe(II)%, with B being a narrow slice of diameter for emphasis. FIGS. 8C and 8D plot performance vs size and saturation magnetization, Ms, with D showing a narrower range of Ms for emphasis. In FIG. 8A, note the cluster of samples around 25-28 nm and ˜40% Fe(II), which displayed the most desirable behavior. In FIGS. 8C and 8D, the favored region is Ms <400, and size between 23 and 28 nm.

FIGS. 9A and 9B. Figure of Merit, χ/Δ, is the peak intensity of χ (H) divided by FWHM. The figure of merit captures both relevant parameters and presents them so that the greatest value (tallest bar) represents the most desirable behavior. In FIG. 9A, the cluster of tall bars is centered around 40% Fe(II), with sizes between 24 and 28 nm. For sizes much larger than 28 nm diameter, performance falls off at 40% Fe(II). In FIG. 9B, the desirable behavior is Ms between 340 and 400 kA/m.

Materials.

1-Octadecene (tech. 90%), oleic acid (tech. 90%), and iron trichloride hexahydrate (ACS, 97.0-102.0%) were obtained from Alfa Aesar. Sodium oleate (>97%) was obtained from Tokyo Chemical Industry CO, LTD. Sodium hydroxide, sodium sulfate (anhydrous), potassium permanganate (99.2%), n-heptane, ethyl acetate and methylene chloride (HPLC grade) were obtained from Fisher Scientific. Hexane (mixture of isomers), chloroform, and acetone were HPLC grade and obtained from Sigma-Aldrich. Ethanol (200 proof) was obtained from Decon Labs. Phosphoric acid (85%), sulfuric acid and concentrated hydrochloric acid were obtained from Macron. 1% oxygen in argon was obtained from Praxair. Water used in any experiment was purified at 18.2 MOhm-cm. The DigiTrol II was obtained from Sigma-Aldrich. SUBA-SEAL® septum were obtained from Chemglass. Poly(maleic anhydride-alt-1-octadecene) (average Mn 30,000-50,000 Da) was obtained from Sigma-Aldrich. mPEG-NH₂ (MW=20,000) was purchased from JenKem.

Synthesis of Iron(III) Oleate.

This is a modification of a published procedure (J. Park, K. An, Y. Hwang, J. G. Park, H.-J. Noh, J.-Y. Kim, J.-H. Park, N.-M. Hwang, and T. Hyeon, “Ultra-large-scale syntheses of monodisperse nanocrystals,” Nature Materials, vol. 3, no. 12, pp. 891-895, November 2004). To a 2-liter three neck round bottom flask equipped with a 1½×⅝ inch Teflon coated magnetic stir bar, was added sodium oleate (147.05 g, 483 mmol) and hexanes (500 mL). The flask was equipped with a glass stopper in the left neck, a SUBA-SEAL® septum with a thermocouple in the right neck, and reflux condenser topped with a schlenk line attachment on the center neck. The mixture was stirred to suspend the sodium oleate, then ethanol (300 mL) was added. The slow (30 seconds) addition of water (60 mL) caused nearly all of the solids to dissolve. The reaction vessel was equipped with a heating mantle and heated to 40° C. with stirring, at which point the sodium oleate had completely dissolved. A solution of iron(III) trichloride hexahydrate (43.518 g, 161 mmol) in water (100 mL) was prepared in a 250 mL Erlenmeyer flask with stirring for about 30 minutes, at which time the iron(III) chloride had completely dissolved. The iron(III) chloride solution was added to the reaction vessel via a funnel with pre-wetted qualitative filter paper (15 cm) and washed in with water (20 mL). The reaction vessel was purged with argon for 1 minute and then heated to a gentle reflux (57° C. internal temperature). The reaction was held at reflux and stirring (500 rpm) was maintained for 4 hours. The heating mantle was then removed and the reaction was allowed to cool to 50° C., then transferred to a 1-liter separatory funnel. The bottom layer was drained and the upper red layer was washed with water (3×150 mL, 10 second shake period). The dark red organic layer was then transferred to a 1-liter Erlenmeyer flask containing anhydrous sodium sulfate (50 g). The solution was swirled occasionally for 10 minutes and then filtered through qualitative filter paper into a 2-liter round bottom flask. The solution was concentrated carefully on a rotary evaporator using a water aspirator for vacuum, first at a water bath temperature of 20° C. and then increased in small increments to 30° C. After solvent removal appeared to have ceased, the vacuum source was switched to high vacuum on the rotary evaporator and concentrating was continued for about 30 minutes at 30° C. bath temperature. After drying on a high vacuum line overnight, the resulting dark red syrup (144.05 g) was deemed to contain 160 mmol of iron(III) oleate and could be divide by mass for use in the nanoparticle synthesis. FT-IR (cm⁻¹) 3600-2500 w, br, 2922 s, 2852 s, 1711 m, 1587 m, 1564 w, 1527 m, 1438 m, 723 w, 613 w, br, 514 w. Elemental analysis of crude product: Found (%): C, 69.56; H, 11.05; N, 0.02; Fe (by ICP), 5.97%. Calculated (%) for Fe₃O(oleate)₇.2oleic acid·4.5H₂O: C, 69.50; H, 11.09; Fe, 5.98.

Synthesis of Iron(III) Oleate as a Solution in 1-Octadecene.

The above standard procedure was modified in the following way: During the work-up, when evaporation of hexane had slowed, 1-octadecene (200 mL) was added and concentrating was continued on a rotary evaporator with high vacuum for about 30 minutes. The iron(III) oleate solution was placed on a high vacuum line overnight. The resulting solution was deemed to contain 160 mmol of iron(III) and 1-octadecene (200 mL) and could be divided by mass for use in the nanoparticle synthesis.

Synthesis of Magnetite Iron Oxide Nanoparticles.

Oleic acid to Fe ratio: 7.3:1. To a 1-liter 3-neck heavy walled round bottom flask with 24/40 joints was added iron(III) oleate (40 mmol, 36.00 g), followed by oleic acid (82.478 g, 304 mmol) and 1-octadecene (200 g). The flask was equipped with a 1½×⅝ inch Teflon coated magnetic stir bar, a glass stopper in the center neck, a SUBA-SEAL® septum with a thermocouple in the right neck, and a bump trap topped with an air condenser and schlenk line attachment on the left neck. A DigiTrol II was used to control the heating of the reaction vessel. The glass joints were sealed with a few drops of 1-octadecene. The reaction was heated to 50° C., held under vacuum and stirred at 450 rpm for 18 hours. The reaction was evacuated and filled with argon five times (holding vacuum for 5 minutes each time) and then purged with argon for 5 minutes. The upper half of the reaction vessel and the necks were wrapped in foil to reduce water condensation. The set point was changed to 110° C. After 15 minutes the internal temperature was 122° C. The controller was set to ramp at 5° C./min and the set point was changed to 318° C. The stir rate was increased to 800 rpm. Purging with argon (40 mL/min) was continued via needle through the septum in the right flask neck to aid in the removal of water vapor into the bump trap. When the temperature reached 318° C. the argon purge through the septum was switched to the schlenk line to maintain an atmosphere of argon. The argon purging line and needle were removed from the septum. Approximately 2 to 3 minutes later, the reaction temperature reached 324° C. Over the next 30 minutes the set point was gradually increased in 2° C. increments to maintain the temperature at 324° C. After 1 hour 40 minutes, since reaching 318° C., the reaction mixture had darkened and finally turned turbid with the color of milk chocolate indicating particle formation. After an additional 30 minutes the set point was changed to 318° C. After the reaction had cooled to 318° C. (about 15 minutes), the addition of 1% oxygen in argon was begun at a flow rate of approximately 140 mL/min via a 16 gauge×6-inch stainless steel needle immersed about 1 inch into the reaction mixture. After 3 hours of 1% oxygen in argon addition the reaction had turned black. The needle was pulled up so the tip of the needle was about 2 inches above the surface of the reaction mixture and the flow rate of 1% oxygen was reduced to about 15 mL/min. The stir rate was reduced to about 450 rpm to prevent possible loss of stirring during the night. The reaction was kept at 318° C. for 34 hours from the time the reaction first reached 318° C. (28.5 hours from the point the 1% oxygen flow was reduced). The heating was turned off using a timer and the reaction was allowed to cool during the night. The cooled reaction mixture had thicken and the reaction mixture was carefully warmed to liquefy the mixture. When the reaction mixture was at 50-60° C. the mixture was transfer to a 500 mL bottle with the aid of hexanes (100 mL) and purged with argon.

The above procedure was repeated multiple times with varying ratios of oleic acid to Fe to provide batches of nanoparticles with a variety of core diameters (representative batches are recorded in Table 1). The range of oleic acid ratios used was 6.5 to 7.8 moles oleic acid per mole iron oleate. The range of oxidation conditions included flow rates of 1% oxygen Argon mix from 15 to 140 mL/min. Examples of different oxidation procedures and the resulting nanoparticle iron (II) composition are provided in Table 2.

Preparation of Samples for TEM Analysis.

A sample for TEM was prepared with 2 mL of reaction mixture added to a 40 mL vial, followed by the addition of hexanes (3 mL) followed by the addition of ethyl acetate (10 mL). The vial was placed on the edge of a FeNdB permanent magnet, Grade N51 (3″×3″×1″) for about 10 minutes. The solution was removed from the resulting black solids and the wash procedure was repeated 2 more times. The black nanoparticles (about 10 mg) were dissolved in chloroform (about 2 mL), sonicated for a few minutes and 10 microliters of the solution were added, in 3-4 drops from a 10 microliter pipette, to a TEM grid (pure carbon film on 200 mesh copper grid, Ted Pella catalog number 01840 F) for TEM imaging and electron diffraction. TEM size analysis, by counting over 1000 particles from micrographs, was performed using the particle size analyzer (PSA r12) plugin available for the Imagej software. Selected area electron diffraction (SAED) patterns were analyzed using the radial integration tool in Imagej.

Nanoparticle Washing Procedure.

A portion of the crude reaction mixture containing nanoparticles (10 mL) was washed by the addition of ethyl acetate (30 mL). The vial was placed on the edge of a FeNdB permanent magnet, Grade N51 (3″×3 ″×1″) for about 10 minutes. The solution was removed from the resulting black solids and the solids were dissolved in heptane (5 mL) with sonication. Ethyl acetate (20 mL) was added and the magnetic separation was repeated. The washing procedure was repeated an additional 2 times. After the last wash, the iron oxide cores were dried under high vacuum for at least 30 minutes to give about 60 mg of iron oxide nanoparticles.

Iron Oxide Nanoparticle Titration Procedure for Determination Fe(II) Content.

A washed and dried sample of nanoparticles (˜60 mg) which was evenly distributed across the bottom of a 40 mL vial, as a fine grained solid to aid digestion, was gently and briefly swirled with concentrated HCl (1.8 mL) under argon. After resting for 30 minutes the digestion was checked by observing any movement of the remaining black flakes to a strong magnetic field. When no movement was observed the digestion was considered complete. The resulting HCl mixture was diluted by the addition of water (35 mL). A 0.05 N solution of potassium permanganate was freshly prepared. The titration was performed using a 1-mL Norm-Ject disposable syringe with a 25 gauge needle and the amount of KMnO₄ solution delivered was determined the mass added (density of 0.05N KMnO₄ was determined during preparation to be 1.0018 g/mL). The end point of the titration was reached when the light pale yellow solution changed to a light clear orange. The oleic acid coating was determined by TGA (˜4%). The remaining mass (˜96%) was assumed to be Fe₃O₄ to calculate the total iron. Alternatively the total iron content could be determined by ICP analysis of the titrated solution.

Phase Transfer of Washed Nanoparticles from Organic to Aqueous Solvent.

Iron oxide nanoparticles were phase transferred from organic to aqueous solvent with a modified poly(maleic anhydride-alt-1-octadecene) (PMAO; Mn˜30,000-50,000 Da) amphiphilic polymer conjugated with monofunctional methoxy-polyethylene glycol amine (m-PEG-NH₂; Mn˜20,000 Da). The modified polymer is hereafter referred to as PMAO-PEG. In a 40 ml glass vial equipped with a silicone septum threaded cap, PMAO-PEG polymer was mixed with a washed and dried nanoparticle sample and dissolved in chloroform at a ratio of about 250 polymer units per nanoparticle. The chloroform volume was adjusted such that the nanoparticle concentration was 1 mg/ml. The nanoparticle and PMAO-PEG mixture in chloroform were sonicated in a water-bath sonicator for 1 hour and then allowed to react by stirring. After 24-48 hours of reaction time, stirring was stopped and chloroform was evaporated using rotary evaporation until a concentrated nanoparticle-polymer solid mixture remained. The nanoparticle-polymer solid mixture was evacuated overnight under high vacuum to ensure complete dryness. To the dried mixture, deionized (DI) water was added such that the iron oxide nanoparticle concentration was about 1 mg/ml. The solution was evacuated and filled with argon four times using a 25-gauge needle inserted through the silicone septum cap. After filling the vial with argon, the nanoparticle-polymer mixture in DI water was sonicated for 90 minutes to yield water-stable PMAO-PEG coated nanoparticles.

Measurement of Differential Magnetic Susceptibility of Iron Oxide Nanoparticles in an AC Field by Magnetic Particle Spectrometer (MPS).

The differential magnetic susceptibility of water-stable PMAO-PEG coated iron oxide nanoparticles was measured with a Magnetic Particle Spectrometer (MPS). The MPS, which can be alternatively known as an “x-space relaxometer,” is an alternating-field magnetometer that simultaneously applies a time-varying magnetic field to excite the magnetization of magnetic particles and measures the signal induced in a receive coil by the nanoparticle magnetization. From the received signal, the nanoparticle's differential susceptibility, χ(H), can be recovered. The technique is described in detail in S. A. Shah, R. M. Ferguson, and K. M. Krishnan, “Slew-rate dependence of tracer magnetization response in magnetic particle imaging,” J. Appl. Phys., vol. 116, no. 16, p. 163910, October 2014. Experiments were performed applying a 25 kHz alternating magnetic field at 20 mT/μ₀ amplitude. The signal is detected by a receive coil with Prior to each measurement, background noise in the MPS transceiver coil was collected and stored. After background collection, a 0.65 ml micro-centrifuge tube containing 0.1 ml of PMAO-PEG coated nanoparticles was inserted in the MPS transceiver coil to measure differential susceptibility. The measurement was repeated three times and the aggregate was plotted. The differential susceptibility (units: m³) was normalized to iron mass (units: gFe) determined from inductively coupled plasma optical emission spectroscopy (ICP-OES). The peak intensity (χ, units: m³ gFe⁻¹) and the full width at half maximum (FWHM or Δ, units: mT) together classified the magnetic response of the nanoparticles in the applied AC field conditions.

Saturation Magnetization Measurement.

The saturation magnetization of iron oxide nanoparticles was measured at room temperature in a Vibrating Sample Magnetometer (VSM). 0.1 ml of PMAO-PEG coated nanoparticles was pipetted in a polycarbonate capsule. The capsule was inserted in a plastic straw that was affixed to the VSM's sample holder. The magnetization hysteresis loop was measured with a maximum field of ±0.2 tesla. The absolute magnetic moment values (A·m²) at 0.2 and −0.2 tesla were averaged to calculate the saturation magnetization.

TABLE 1
Exemplary Iron Oxide Nanoparticles

| | | | | | | | | Fe(II) % w/ | Fe(II) % w/ | |
|---|---|---|---|---|---|---|---|---|---|---|
| | | | | | | | Fe(II) % | 1.4 nm | 2 nm | |
| | TEM- | TEM- | MPS- | Sig-vs- | MPS- | fwhm-vs- | from | maghemite | maghemite | Ms |
| Core | dN | sigma | m\hat{ }3/gFe | Resovist | FWHM | Resovist | titration | Shell | Shell | [kA/m] |
| 9-107 | 18.2 | 0.06 | | 0.00 | | 0% | 19.2% | 31.7% | 37.2% | |
| 9-115 | 26.2 | 0.05 | 1.70E−05 | 3.27 | 7.9 | 70% | 24.6% | 34.5% | 38.3% | 375 |
| 9-122c | 26.6 | 0.08 | 9.00E−05 | 1.73 | 12.8 | 113% | 29.3% | 39.8% | 43.8% | 305 |
| 9-123C | 29.2 | 0.07 | 1.20E−05 | 2.31 | 11.7 | 104% | 29.6% | 40.1% | 43.9% | 345 |
| 9-129B | 27.4 | 0.05 | 1.90E−05 | 3.65 | 8.7 | 77% | 28.6% | 36.8% | 40.6% | 372 |
| 9-133 | 20.5 | 0.07 | 1.82E−05 | 3.50 | 7.9 | 70% | 20.9% | 32.5% | 37.8% | 412 |
| 9-145 | 26.9 | 0.06 | 1.86E−05 | 3.58 | 9.5 | 84% | 25.3% | 35.2% | 38.9% | 415 |
| 9-156 | 27.4 | 0.07 | | | | | 30.7% | 42.4% | 46.8% | 364 |
| 9-160 | 21.5 | 0.08 | 1.85E−05 | 3.56 | 6.4 | 57% | 24.2% | 36.8% | 41.9% | 375 |
| 9-168 | 21.9 | 0.07 | 2.07E−05 | 3.98 | 5.9 | 52% | 21.6% | 32.6% | 37.0% | 370 |
| 9-172 | 24.4 | 0.05 | 1.96E−05 | 3.77 | 6.9 | 61% | 25.0% | 37.5% | 42.0% | 395 |
| 9-177 | 25.3 | 0.08 | 1.63E−05 | 3.13 | 8.0 | 71% | 26.1% | 37.1% | 41.4% | 377 |
| 9-186 | 24.4 | 0.09 | 2.14E−05 | 4.12 | 6.4 | 57% | 27.9% | 40.2% | 45.0% | 401 |
| 9-187 | 24.1 | 0.07 | 2.43E−05 | 4.67 | 6.4 | 57% | 25.3% | 36.6% | 41.1% | 402 |
| 11-16 | 24.9 | 0.07 | 2.60E−05 | 5.00 | 5.8 | 51% | 28.5% | 40.8% | 45.5% | 377 |
| 11-17 | 28.1 | 0.08 | 3.33E−05 | 6.40 | 5.2 | 46% | 30.0% | 41.1% | 45.3% | 364 |
| 11-17 | 28.1 | 0.08 | 3.49E−05 | 6.71 | 4.8 | 42% | 30.0% | 41.1% | 45.3% | |
| 11-18 | 25.1 | 0.06 | 2.78E−05 | 5.35 | 5.4 | 48% | 28.4% | 40.5% | 45.2% | 390 |
| 11-22 | 24.3 | 0.07 | 1.99E−05 | 3.83 | 7.3 | 65% | 26.0% | 37.5% | 42.1% | 339 |
| 11-23 | 25.5 | 0.08 | 2.75E−05 | 5.29 | 6.7 | 59% | 27.5% | 39.0% | 43.4% | 390 |
| 11-25 | 26.5 | 0.06 | 1.27E−05 | 2.44 | 6.4 | 57% | 35.6% | 49.8% | 55.2% | 239 |
| 11-26 | 32.2 | 0.08 | 1.57E−05 | 3.02 | 10.0 | 88% | 29.5% | 36.8% | 42.1% | 323 |
| 11-27 | 32 | 0.07 | 1.61E−05 | 3.10 | 9.1 | 81% | 28.8% | 37.9% | 41.2% | 318 |
| 11-44 | 27.6 | 0.08 | | | | | 66.0% | 91.0% | 100.4% | |
| 11-50 | 20.9 | 0.07 | 1.24E−05 | 2.68 | 9.2 | 81% | 26.1% | 40.2% | 46.0% | 370 |
| 11-51 | 21 | 0.06 | 2.05E−05 | 3.94 | 7.2 | 64% | 25.8% | 39.6% | 45.4% | 375 |
| 11-52 | 28.5 | 0.07 | 1.82E−05 | 3.50 | 10.6 | 94% | 27.4% | 37.4% | 41.1% | 378 |
| 11-53 | 29.7 | 0.06 | 2.44E−05 | 4.69 | 7.9 | 70% | 24.0% | 32.3% | 35.4% | 395 |
| 11-64 | 24.4 | 0.05 | 1.87E−05 | 3.60 | 5.0 | 44% | 35.2% | 50.7% | 56.8% | 255 |
| 11-65 | 25.4 | 0.06 | 1.47E−05 | 2.83 | 5.0 | 44% | 40.8% | 57.9% | 64.5% | 255 |
| 11-66 | 30.2 | 0.09 | 1.94E−05 | 3.73 | 10.1 | 89% | 29.6% | 39.6% | 43.3% | 385 |
| 11-67 | 30.9 | 0.1 | 2.07E−05 | 3.98 | 10.0 | 88% | 30.1% | 40.0% | 43.6% | 393 |
| 11-71 | 25.9 | 0.07 | 3.13E−05 | 6.02 | 6.3 | 56% | 29.6% | 41.7% | 46.4% | 405 |
| 11-76 | 23.6 | 0.06 | 1.63E−05 | 3.13 | 6.3 | 56% | 42.9% | 62.7% | 70.5% | 280 |
| 11-77 | 21.2 | 0.05 | 1.57E−05 | 3.02 | 6.5 | 58% | 38.5% | 58.9% | 67.3% | 295 |
| 11-80 | 23.2 | 0.07 | 1.29E−05 | 2.48 | 5.6 | 50% | 41.4% | 60.9% | 68.7% | 227 |
| 11-81 | 22.5 | 0.07 | 1.60E−05 | 3.08 | 6.0 | 58% | 36.9% | 55.0% | 62.3% | 267 |

TABLE 2
Exemplary Nanoparticle Synthesis Conditions and Resulting Properties

| | oxidation part 1 | | oxidation part 2 | | | | core | |
|---|---|---|---|---|---|---|---|---|
| | 1% O2 in Ar | | 1% O2 in Ar | | TEM- | | Fe(II) % | |
| | flow rate | time | flow rate | time | dN | TEM- | from | Ms |
| Core | [ml/min] | [hrs] | [ml/min] | [hrs] | [nm] | sigma | titration | [kA/m] |
| 9-187 | 110 | 3 | 25 | 18 | 24.1 | 0.07 | 25.3 | 402 |
| 11-17 | 130 | 1.33 | 4 | 22 | 28.1 | 0.08 | 30.0 | 364 |
| 11-50 | 110 | 1 | 4 | 22 | 20.9 | 0.07 | 26.1 | 370 |
| 11-67 | 2.6 | 19.5 | n/a | n/a | 30.9 | 0.1 | 30.1 | 393 |

While illustrative embodiments have been illustrated and described, it will be appreciated that various changes can be made therein without departing from the spirit and scope of the invention.

1. A plurality of iron oxide nanoparticles, each iron oxide nanoparticle comprising a core comprising an iron oxide inner core comprising an intermediate phase between stoichiometric FeO (100% of total iron is iron (II)) and stoichiometric Fe₃O₄ (33.3% of total iron is iron (II), the balance iron (III)), the inner core composed of iron (II) in an amount of from 34-50% of the total iron.

2. (canceled)

3. The plurality of iron oxide nanoparticles of claim 1, wherein the core further comprises a shell of iron oxide with a thickness of 0.7 nm to 2 nm surrounding the inner core.

4. The plurality of iron oxide nanoparticles of claim 3, wherein the shell is maghemite (Fe₂O₃).

5. The plurality of iron oxide nanoparticles of claim 1, further comprising a coating layer disposed on an exterior surface of the core.

6-14. (canceled)

15. The plurality of iron oxide nanoparticles of claim 1, wherein the plurality of iron oxide nanoparticles have a narrow distribution of diameters defined by a geometric standard deviation of 1.2 or less.

16. The plurality of iron oxide nanoparticles of claim 1, wherein the plurality of nanoparticles have a median diameter range of 10 nm to 40 nm.

17. The plurality of iron oxide nanoparticles of claim 1, wherein the plurality of iron oxide nanoparticles have a mass magnetization of 67 to 111 A·m²/kg Fe.

18-19. (canceled)

20. The plurality of iron oxide nanoparticles of claim 1, wherein the plurality of iron oxide nanoparticles have a saturation magnetization of 250 to 415 kA/m.

21. (canceled)

22. The plurality of iron oxide nanoparticles of claim 1, wherein the plurality of iron oxide nanoparticles has an intensity greater than 2.0×10⁻⁵ m³/gFe and full width at half maximum less than 6.5 mT/μ₀ as measured by a magnetic particle spectrometer at 25 kHz excitation frequency and 20 mT/μ₀ amplitude.

23. The plurality of iron oxide nanoparticles of claim 1, wherein the plurality of iron oxide nanoparticles are configured for use as magnetic particle imaging tracers.

24. (canceled)

25. The plurality of iron oxide nanoparticles of claim 1, wherein the magnetic tracers are configured for use in a magnetic imaging technique selected from the group consisting of magnetic particle imaging and magnetic resonance imaging.

26. A method, comprising applying a magnetic field to a plurality of iron oxide nanoparticles according to claim 1.

27-44. (canceled)

45. A plurality of iron oxide nanoparticles, each iron oxide nanoparticle comprising a core of iron oxide, wherein
the plurality of iron oxide nanoparticles has an iron (II) content of 25-37% of the total iron content; and
wüstite (FeO) is visible in a select area electron diffraction image obtained by transmission electron microscopy.

46. The plurality of iron oxide nanoparticles of claim 45, wherein the core further comprises a shell of iron oxide with a thickness of 0.7 nm to 2 nm surrounding the inner core.

47. The plurality of iron oxide nanoparticles of claim 46, wherein the shell is maghemite (Fe₂O₃).

48. The plurality of iron oxide nanoparticles of claim 45, further comprising a coating layer disposed on an exterior surface of the core.

49. The plurality of iron oxide nanoparticles of claim 45, wherein the plurality of iron oxide nanoparticles have a narrow distribution of diameters defined by a geometric standard deviation of 1.2 or less.

50. The plurality of iron oxide nanoparticles of claim 45, wherein the plurality of nanoparticles have a median diameter range of 10 nm to 40 nm.

51. The plurality of iron oxide nanoparticles of claim 45, wherein the plurality of iron oxide nanoparticles have a mass magnetization of 67 to 111 A·m²/kg Fe.

52. The plurality of iron oxide nanoparticles of claim 45, wherein the plurality of iron oxide nanoparticles have a saturation magnetization of 250 to 415 kA/m.

53. The plurality of iron oxide nanoparticles of claim 45, wherein the plurality of iron oxide nanoparticles has an intensity greater than 2.0×10⁻⁵ m³/gFe and full width at half maximum less than 6.5 mT/μ₀ as measured by a magnetic particle spectrometer at 25 kHz excitation frequency and 20 mT/μ₀ amplitude.

54. The plurality of iron oxide nanoparticles of claim 45, wherein the plurality of iron oxide nanoparticles are configured for use as magnetic particle imaging tracers.

55. The plurality of iron oxide nanoparticles of claim 45, wherein the magnetic tracers are configured for use in a magnetic imaging technique selected from the group consisting of magnetic particle imaging and magnetic resonance imaging.

56. A method, comprising applying a magnetic field to a plurality of iron oxide nanoparticles according to claim 45.
